(19) United States
(12) Patent Application Publication
(10) Pub. No.: US 2019/0136256 A1
(43) Pub. Date: May 9, 2019

(54) METHODS OF CONTROLLING VEGETATIVE GROWTH AND FLOWERING TIMES BY MODULATING PHOSPHOENOLPYRUVATE SHUNT BETWEEN SHIKIMATE AND GLYCOLYSIS PATHWAYS

(71) Applicant: UT-Battelle, LLC, Oak Ridge, TN (US)

(72) Inventors: Jay CHEN, Oak Ridge, TN (US); Wellington MUCHERO, Oak Ridge, TN (US); Gerald A. TUSKAN, Oak Ridge, TN (US); Anthony Christian BRYAN, Oak Ridge, TN (US)

(21) Appl. No.: 16/183,074

(22) Filed: Nov. 7, 2018

Related U.S. Application Data
(60) Provisional application No. 62/582,606, filed on Nov. 7, 2017.

(51) Int. Cl. C12N 15/82

(52) U.S. Cl. CPC C12N 15/8279; C12N 2310/14; C12N 2310/20; C12N 15/827

(57) ABSTRACT

Disclosed herein are methods of producing plants with preferred levels of vegetative growth periods, flowering times, and resistance to stress and pathogens; and uses of such plants. The inventors have identified that the ANGUSTIFOLIA(AtAN)/CtBP gene is a major determinant of the carbon allocation between the Shikimate Pathway and the Salicylic Acid (SA) and Jasmonic Acid(JA)/Ethylene pathway. Plants with modulated growth periods, flowering times, and resistance to stress/pathogen characteristics, based on modulation of the expression or activity of the ANGUSTIFOLIA (ATAN)/CTBP gene, have divergent uses including pulp and paper production, bioproduct production, and as pathogen-resistant crops.

### CROSS REFERENCE TO RELATED APPLICATION

This application claims the benefit of priority from U.S. Provisional Application No. 62/582,606, filed Nov. 7, 2017, the entire contents of which are incorporated herein by reference.

### STATEMENT REGARDING FEDERALLY SPONSORED RESEARCH OR DEVELOPMENT

This invention was made with government support under a research project supported by Prime Contract No. DE-AC05-000R22725 awarded by the U.S. Department of Energy. The government has certain rights in this invention.

### INCORPORATION BY REFERENCE OF SEQUENCE LISTING

The Sequence Listing in the ASCII text file, named as 33715_Seq_ST25.txt of 19 KB, created on Oct. 17, 2018, and submitted to the United States Patent and Trademark Office via EFS-Web, is incorporated herein by reference.

### BACKGROUND OF THE DISCLOSURE

In plants, resistance to pathogen infection is accomplished through protective physical barriers and a diverse array of antimicrobial chemicals and proteins. Many of these antimicrobial compounds are part of an active defense response, and their rapid induction is contingent on the plant's ability to recognize and respond to an invading pathogen. Disease resistance in plants is regulated by multiple signal transduction pathways in which salicylic acid (SA), jasmonic acid (JA), and ethylene (ET) function as key signaling molecules.

The apparent antagonism between salicylic acid (SA) and Jasmonic acid (JA)/ethylene (ET) signaling is one of the biggest biological questions that remain unanswered. This phenomenon has major implications in the tradeoff between growth and defense against pathogens where numerous studies have demonstrated that triggering immune responses often resulted in growth inhibition and major yield losses just as detrimental as the disease itself in many plant species.

The Shikimate Pathway (shikimic acid pathway) is a seven step metabolic route used by bacteria, fungi, algae, parasites and plants for the biosynthesis of aromatic amino acids (phenylalanine, tyrosine, and tryptophan). This pathway is not found in animals, which require these amino acids, hence the products of this pathway represent essential amino acids that must be obtained from bacteria or plants (or animals which eat bacteria or plants) in the animal's diet.

Bacteria spend >90% of their total metabolic energy on protein biosynthesis. Consequently, the bacterial shikimate pathway serves almost exclusively to synthesize the aromatic amino acids. In contrast, higher plants use these amino acids not only as protein building blocks but also, and in even greater quantities, as precursors for a large number of secondary metabolites, among them plant pigments, compounds to defend against insects and other herbivores, UV light protectants, and, most importantly, lignin. Under normal growth conditions, 20% of the carbon fixed by plants flows through the shikimate pathway.

The Shikimate Pathway is remarkably conserved across prokaryotes and eukaryotes to the extent that heterologous transformation of genes associated with the pathway has been successful across highly divergent organisms including bacteria, fungi and plants. Being the only source of aromatic amino acid-precursors for the phenylpropanoid, tryptophan, tyrosine and flavonoid pathways, as well as shuttling between 30-50% of all fixed carbon, the shikimate pathway is a crucial source of structural, defense, light harvesting and hormone signaling molecules essential for plant survival. Additionally, since humans and animals cannot synthesize aromatic amino acids, they are dependent on this pathway as the only dietary source of these essential amino acids. As such, the shikimate pathway has been extensively studied across highly divergent taxa for economic, nutritional and medicinal reasons. In plants, one of the most characterized steps in the shikimate pathway is the sixth reaction that is catalyzed by the enzyme 5-enolpyruylshikimate 3-phosphate synthase (EPSP) synthase. This enzyme catalyzes the conversion of shikimate 3-phosphate to 5-enolpyruylshikimate 3-phosphate and is the target of the herbicide glyphosate. Isoforms of this enzyme derived from some microbes are naturally resistant to glyphosate and have been used extensively in heterologous transformation to create herbicide resistant plants. No other roles have been assigned for EPSP synthase outside of catalysis in the Shikimate Pathway. Shikimate pathway is described in detail by Hermann, K M (Plant Cell., 1995 July; 7(7):907-919).

The ANGUSTIFOLIA(AtAN)/CtBP is related to the animal C-terminal Binding Protein (CtBP/BARS), which is known to function as a corepressor. Plant homologs of CtBP are monophyletic compared to animal homologs and contain an additional C-terminal extension not seen in animal CtBP. The Arabidopsis thaliana homolog has been previously characterized and named Angustifolia (AN). Null an mutants in Arabidopsis (AtAN) display narrow cotyledons and rosette leaves, reduced growth and delayed flowering. This narrow leaf phenotype attributed to misregulation of polar elongation in leaf epidermal cells (Tsuge, T, et al., Development, 122:1589-1600 (1996)). AtAN has been further demonstrated to regulate cortical microtubule arrangements in epidermal cells (Kim, G-T, et al., The EMBO J 21:1267-1279 (2002)). This association is of great interest to cell wall chemistry in that previous analysis demonstrated the involvement of cortical microtubules in regulating cellulose microfibril insertion in the cell wall through determining the insertion of the cellulose synthase complexes into the cell membrane (Crowell, E, et al., The Plant Cell, 21:1141-1154 (2009)).

The present inventors' identification and manipulation of genes regulating carbon flow between the Shikimate pathway and other metabolic pathways is critical both for regulating vegetative growth and flowering of plants, and for modulating plant resistance to disease.

### BRIEF SUMMARY OF THE DISCLOSURE

In one aspect, this disclosure provides a method comprising modulating the expression of the ANGUSTIFOLIA gene in a plant. In some embodiments, modulating the expression of the ANGUSTIFOLIA gene comprises altering the expression of the ANGUSTIFOLIA gene. In some embodiments, the term “altering” refers to decreasing the expression of a gene, for example, by downregulating the gene expression (e.g., using antisense, siRNA, miRNA, etc.), or by inactivation of the gene by CRISPR, TALEN, ZNF, Cre-lox targeting, etc.). In some embodiments, the term “altering” refers to increasing the expression of a gene (e.g., by introducing a nucleic acid encoding for the gene in a suitable expression vector, etc.).

In some embodiments, modulating the expression of the ANGUSTIFOLIA gene in a plant comprises inactivating the ANGUSTIFOLIA gene in the plant.

In some embodiments, the inactivation of the ANGUSTIFOLIA gene in a plant is achieved by introducing a nucleic acid inhibitor of the ANGUSTIFOLIA gene to the plant.

In some embodiments, the nucleic acid inhibitor is selected from the group consisting of an antisense RNA, a small interfering RNA, an RNAi, a microRNA, an artificial microRNA, and a ribozyme.

In some embodiments, the inactivation of the ANGUSTIFOLIA gene is achieved by genome editing.

In a specific embodiment, said genome editing comprises CRISPR-mediated genome editing. In some embodiments, the CRISPR-mediated genome editing comprises introducing into the plant a first nucleic acid encoding a Cas9 nuclease, a second nucleic acid comprising a guide RNA (gRNA), wherein said NA is specific to the ANGUSTIFOLIA gene.

In some embodiments, the modulation of the ANGUSTIFOLIA gene expression comprises introducing an exogenous nucleic acid encoding an ANGUSTIFOLIA gene into the plant. In some embodiments, the exogenous nucleic acid is stably transfected or transformed into the plant genome. In some embodiments, the exogenous nucleic acid is expressed transiently.

In some embodiments, the plant used in the present methods is a member of the genus selected from the group consisting of Acer, Afzelia, Arabidopsis, Betula, Brassica, Eucalyptus, Fagus, Fraxinus, Glycine, Gossypium, Jatropha, Juglans, Linum, Lycopersicon, Medicago, Micropus, Populus, Prunus, Quercus, Salix, Solanum, Tectona, Trifolium, Agrostis, Avena, Festuca, Hordeum, Lemna, Miscanthus oryza, Panicum, Pennisetum, Phalaris, Phleum, Poa, Saccharum, Secale, Sorghum, Triticum, Zea, Zoysia, Abies, Picea and Pinus.

In a specific embodiment, the plant used in the present methods is selected from the group consisting of Festuca arundinacea, Miscanthus giganteus, Miscanthus sinensis, Miscanthus sacchariflorus, Panicum virgatum, Pennisetum purpureum, Phalaris arundinacea, Populus balsamifera, Populus deltoides, Populus tremuleides, Populus tremula, Populus alba, Populus maximowiczii, Saccharum officinarum, Saccharum ravennae, Secale cereale, Sorghum almum, Sorghum halcapense, and Sorghum vulgare.

Another aspect of this invention is directed towards a plant, in which the ANGUSTIFOLIA gene is modulated and altered as compared to a plant in which the ANGUSTIFOLIA gene is not modulated (a control plant). In some embodiments, the plant in which the ANGUSTIFOLIA gene is modulated is a member of the genus selected from the group consisting of Acer, Afzelia, Arabidopsis, Betula, Brassica, Eucalyptus, Fagus, Fraxinus, Glycine, Gossypium, Jatropha, Juglans, Lycopersicon, Medicago, Micropus, Populus, Prunus, Quercus, Salix, Solanum, Tectona, Trifolium, Agrostis, Avena, Festuca, Hordeum, Lemna, Lolium, Milium, Miscanthus oryza, Panicum, Pennisetum, Phalaris, Phleum, Poa, Saccharum, Secale, Sorghum, Triticum, Zea, Zoysia, Abies, Picea and Pinus. In a specific embodiment, the plant in which the ANGUSTIFOLIA gene is modulated is selected from the group consisting of Festuca arundinacea, Miscanthus giganteus, Miscanthus sinensis, Miscanthus sacchariflorus, Panicum virgatum, Pennisetum purpureum, Phalaris arundinacea, Populus balsamifera, Populus deltoides, Populus tremuloides, Populus tremula, Populus alba, Populus maximowiczii, Saccharum officinarum, Saccharum ravennae, Secale cereale, Sorghum almum, Sorghum halcapense, and Sorghum vulgare

A different aspect of this disclosure provides a method for bioproduct production, comprising subjecting a plant in which the ANGUSTIFOLIA gene is modulated to a bioproduct conversion process. In some embodiments, the bioproduct is selected from the group consisting of a bioenergy product, a biomaterial, a biopharmaceutical and a biocosmetics. In some embodiments, the bioenergy product is ethanol and the bioproduct conversion process is an ethanol fermentation process. In another embodiment, the bioproduct is selected from the group consisting of ethanol, biodiesel, biogas, bioplastics, biofoams, biorubber, biocomposites, and biofibres.

Another aspect of this disclosure provides a method for production of pulp or paper, comprising producing pulp or paper from a plant in which the ANGUSTIFOLIA gene is modulated. In a specific embodiment, the ANGUSTIFOLIA gene in a plant is inactivated.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1E. The Phosphoenol pyruvate (PEP) shunt between the Shikimate and Glycolysis pathways. (A) Phosphoenol pyruvate (PEP) is used as a precursor in either the Shikimate pathway (leading to cell wall biosynthesis), or the Pyruvate/Malate biosynthesis pathway (leading to Jasmonic acid (JA)/ethylene (ET) production and disease resistance). (B)-(E) When ANGUSTIFOLIA (AtAN)/CtBP gene in Arabidopsis is knocked out by T-DNA insertion (designated Atan-t1), cell wall biosynthesis genes are upregulated in the mutant plants (B), whereas pyruvate/malte biosynthesis (C), disease (D), and ethylene signaling (E) genes are downregulated.

FIG. 2A-2B. Transcriptional changes of canonical pathway marker genes for cell wall (A), pyruvate, jasmonate-mediated defense and ethylene biosynthesis (B) pathways in response to loss of function of the Angustifolia gene in Arabidopsis. Blue bars represent the expression of stated genes in the loss-of-function mutant of the Angustifolia gene in Arabidopsis, and the red bars represent data from wild-type plants.

FIG. 3. Direct binding of MYB46 promoter by the ANGUSTIFOLIA (AN) transcriptional repressor. Biotin labeled MYB46 promoter (lane 1 indicates the position of unbound MYB46 promoter) was incubated with GST alone (lane 2) or GST-AN (GST tagged ANGUSTIFOLIA protein) (lanes 3 and 4) and run on a separating polyacrylamide gel. A specific supershift (arrow) was detected when the MYB46 promoter was incubated with GST-AN (lane 3) and this interaction was competed out by a 100-times excess of non-labeled MYB46 promoter (lane 4).

FIG. 4A-4B. The nuclear accumulation of AN. (A) Subcellular localization analysis of the partial nuclear localization of AN in the Arabidopsis protoplasts. The overlapping of AN-YFP (green) and mCherry-VirD2NLS (nuclear marker, red) is indicated as yellow color. Scale bar: 5 μm. (B) Immunoblot analysis of the nuclear accumulation of AN. AN-Myc, UGPase (cytosolic marker), histone H3 (nuclear marker) are examined in cytosolic (C) and nuclear (IN) proteins.

FIG. 5A-5C. TDP1 enhances the nuclear accumulation of AN. (A) Yeast two-hybrid analysis of the interaction between AN and TDP1. Left panel: domain structures of truncated TDP1 are displayed. “+” indicates interaction; “−” indicates no interaction. Right panel: yeast two-hybrid analysis of AN and TDP1 interaction, AN was fused with Gal4 activation domain (AD). Full-length and truncated TDP1 were fused with Gal4 DNA binding domain (BD). SD-T-L, indicates SD plate without Leu and Trp. SD-T-L-H-A, indicates SD plate without Leu, Trp, His, and Ade. (B) Co-localization analysis of AN with TDP1. CFP-SHY2 (blue) is a nuclear marker. Scale bar: 10 μm. (C) Enhancement of nuclear accumulation of AN by TDP1. AN and TDP1 were blotted using anti-Myc and anti-HA antibodies, respectively.

FIG. 6A-6F. AN has transcriptional repressor activity and directly targets MYB46. (A) Transactivation analysis of the repressor activity of AN. Left scheme displays the three vectors used in transactivation analyses: reporter construct containing Gal4 binding site and LexA binding site upstream of GUS reporter gene; transactivation construct expressing LexA binding domain (LD) fused VP16; effector construct expressing Gal4 binding domain (GD) fused AN. GUS activity in individual samples was normalized against luciferase activity (GUS/LUC). (B) Transactivation analysis of the activator activity of AN. Left scheme displays the two vectors used in transactivation analysis: reporter construct contains one Gal4 binding site upstream of GUS reporter gene; effector construct expressing GD fused AN. GD fused VP16 (GD-VP16) was used as a positive control. (C) Bar graphs of published RNA-sect data (Bryan et al., 2018) showing lignin biosynthesis- and immunity-related genes with opposite expression patterns in an-1 mutants. (D) μChIP-PCR analyses of AN association with promoters of genes up-regulated in an-1 (b, MYB46, MYB58, MYB63, MYB55, MYB20, MYB103, NAC073) and genes down-regulated in an-1 (c, WRKY33, WRKY40, WRKY53, WRKY26, or WRKY22). Two negative controls are shown: no antibody control (−) and protoplasts without expression of AN-Myc (untransfected control). +, indicates reactions with anti-Myc antibody. (E) μChIP-qPCR analysis of the association of AN with MYB46 promoter. The promoter region of ACTIN7 (AT5G09810) was amplified simultaneously as a negative control. ChIP reactions without antibody (no ab) were performed to evaluate the specificity of anti-Myc antibody. (F) EMSA analysis showing the direct binding of AN to the MY1346 promoter. The 250-bp MYB46 promoter (−460 to −210 from start codon) was labeled with biotin as the probe (P-MYB46-biotin). The competition assay was performed using 100 times unlabeled MYB46 promoter DNA (100× P-MYB46). For bar graphs, error bars represent SE of three biological replicates. P value comparison was calculated using two tailed students t-tests (**P<0.01, *P<0.05, ns P>0.05).

FIG. 7A-7D. TDP1 enhances the transcriptional function of AN. (A)-(C) Transactivation analyses of the AN transcriptional repression on MYB46. (A) The vectors used in transactivation analyses (B) and (C): reporter construct containing: MYB46 promoter and GUS reporter gene; transactivation construct expressing VND6; effector construct expressing AN. (D) μChIP-qPCR analysis demonstrating the enhancement of association of AN with MYB46 promoter by TDP1. Error bars represent SE of three biological replicates. P value comparison was calculated using two tailed students t-tests (**P<0.01, *P<0.05, ns P>0.05).

FIG. 8A-8H. AN releases transcriptional repression of WRKY33 by TDP1. (A)-(B) Negative co-expression of TDP1 and WRKY33 across various tissues (A) and pathogen infections (B). Gene expression data were obtained from AtGenExpress Visualization Tool (AVT), (C)-(D) qRT-PCR results showing effects of AN and TDP1 on the expression of WRKY33, ACS6, ACS2, and ERF1A. (C) transcript levels of AN and TDP1. (D) transcript levels of WRKY33, ACS6, ACS2, and ERF1A. (E) μChIP-qPCR analysis of TDP1 association with WRKY33, ACS6, ACS2, and ERF1A promoters. (F)-(G) Transactivation analysis of TDP1 repression of WRKY33. (F) A scheme of two constructs used in the transactivation analysis: reporter constructs containing 35S promoter, WRKY33 promoter, and GUS reporter gene; effector construct expressing TDP1(G). (H) μChIP-qPCR analysis demonstrating that AN reduces TDP1 association with WRKY33 promoter. Error bars represent SE of three biological replicates. P value comparison was calculated using two tailed students t-tests (**P<0.01, *P<0.05, ns P=0.05).

FIG. 9A-9E. AN regulates the tradeoff between lignin biosynthesis and B. cinerea defense. (A) A scheme of proposed model of AN's nuclear function. JA, jasmonate, ET, ethylene; SA, salicylic acid. (B) an-1 mutant and AN overexpression transgenic plants, growth phenotypes of Col-0, an-1, and an-1 35S:AN. (C) qRT-PCR analysis of AN expression levels in transgenic plants. (D) Lesion diameter changes of Col-0, an-1, and an-1 35S:AN from 0 to 72 hpi with B. cinerea inoculation. (E) Phloroglucinol-HCl staining of 7-day-old roots from Col-0, an-1 and an-1 35S:AN. Scale bar: 500 μm.

FIG. 10. Bar graph of transcriptional changes of MYB46 and WRKY33 during infections with B. cinerea, Pseudomonas syringae (P. syringae), Phytophthora infestans (P. infestans), Erysiphe orontil (E. orontii), Hyaloperonospora arabidopsidis (H. arabidopsidis), herbivore stress (Myzus persicaere; M. persicaere) and flg22 treatment. Log₂ of fold changes of MYB46 and WRKY33 were obtained from Arabidopsis eFP Browser (University of Toronto, Canada).

### DETAILED DESCRIPTION OF THE DISCLOSURE

### Definitions

As used herein, the term “about” refers to a variation within approximately ±10% from a given value.

As used herein, “allelic variants” are alternative forms of the same gene or genetic locus. Each allelic variant has a distinct nucleic acid sequence at the locus of interest. For example, the inventors have discovered two allelic variants of the ANGUSTIFOLIA (ATAN)/CTBP gene, the nucleic acid sequences of which differ from each other by at least one nucleotide. The nucleic acid sequence of the ANGUSTIFOLIA (ATAN)/CTBP gene is set forth in SEQ ID NO: 2. An allelic variant of ANGUSTIFOLIA (ATAN)/CTBP can encode the amino acid sequence as set forth in SEQ ID NO: 1, or an amino acid sequence with at least 60% sequence identity, e.g., 60%, 65%, 70%, 75%, 78%, 80%, 81%, 82%, 83%, 84%, 85%, 86%, 87%, 88%, 89%, 90%, 95%, 97%, 98% or 99% sequence identity, to the amino acid sequence set forth in SEQ ID NO: 1. Sequence identity refers to the percent of exact matches between the amino acids of two sequences which are being compared. Where one allelic variant encodes a truncated protein relative to the protein encoded by another allelic variant, percent identity can be determined by comparing the amino acid sequences of the variants along the length of the shorter protein.

This disclosure also provides homologs of the polypeptide encoded by ANGUSTIFOLIA gene. An ANGUSTIFOLIA (ATAN)/CTBP homolog can be a homolog, ortholog or variant of the polypeptide having the amino acid sequence set forth in SEQ ID NO: 1. For example, an ANGUSTIFOLIA (ATAN)/CTBP homolog can have an amino acid sequence with at least 60% sequence identity, e.g., 60%, 65%, 70%, 75%, 78%, 80%, 81%, 82%, 83%, 84%, 85%, 86%, 87%, 88%, 89%, 90%, 95%, 97%, 98% or 99% sequence identity, to the amino acid sequence set forth in SEQ ID NO: 1.

In some embodiments, a homolog of ANGUSTIFOLIA (ATAN)/CTBP is a functional homolog. A functional homolog is a polypeptide that has sequence similarity to SEQ ID NO: 1 and that carries out one or more of the biochemical or physiological function(s) of the polypeptide of SEQ ID NO: 1. A functional homolog may be a natural occurring polypeptide and the sequence similarity may be due to convergent or divergent evolutionary events. As such, functional homologs are sometimes designated in the literature as homologs or orthologs or paralogs. Variants of a naturally occurring functional homolog, such as polypeptides encoded by mutants of a wild type coding sequence, may themselves be functional homologs. Functional homologs can also be created via site-directed mutagenesis of the coding sequence for a cell wall-modulating polypeptide or by combining domains from the coding sequences for different naturally-occurring cell wall-modulating polypeptides (“domain swapping”). The term “functional homolog” can also be applied to the nucleic acid that encodes a functionally homologous polypeptide.

An “altered level of gene expression” refers to a measurable or observable change in the level of expression of a transcript of a gene, or the amount of the encoded polypeptide, relative to a control plant or plant cell under the same conditions (e.g., as measured through a suitable assay such as quantitative RT-PCR, a Northern blot, a Western blot or through an observable change in phenotype, chemical profile or metabolic profile). An altered level of gene expression can include up-regulated or down-regulated expression of a transcript of a gene or polypeptide relative to a control plant or plant cell under the same conditions. Altered expression levels can occur under different environmental or developmental conditions or in different locations than those exhibited by a plant or plant cell in its native state. An altered level of gene expression of a particular gene can be 5%, 10%, 20%, 30%, 40%, 50%, 60%, 70%, 80%, 90%, 95%, 100% or more relative to the expression of the gene in a control plant or plant cell under the same conditions.

As used herein, the term “bioproduct” refers to products made from biological materials. In some embodiments, “bioproducts” include bioenergy products (e.g., liquid fuels (such as ethanol and biodiesel), solid biomass for combustion to generate heat and power, and gaseous fuel (such as biogas and syngas) which can be used to generate heat and power). In some embodiments, “bioproducts” include biomaterials (e.g., bioplastics from plant oils and sugars, biofoams and biorubber from plant oils and latex, biocomposites manufactured from agricultural (e.g., hemp, flax, kenaf) and forestry biofibres, used, for example, in the production of automobile door panels and parts). In some embodiments, “bioproducts” include biochemicals (e.g., industrial materials including, but not limited to, basic and specialty chemicals and resins, including paints, lubricants and solvents), biopharmaceuticals (e.g., natural source medicinal compounds), and biocosmetics (e.g., soaps, body creams, shampoos, lotions, herbal extracts)).

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

As used herein, the term “CRISPR” refers to a RNA-guided endonuclease comprising a nuclease, such as Cas9, and a guide RNA that directs cleavage of the DNA by hybridizing to a recognition site in the genomic DNA.

The term “disease resistance” or “pathogen resistance” refers to the ability to maintain a desirable phenotype upon exposure to infection, relative to a nontransgenic (wild type) plant. The level of resistance can be determined by comparing the physical characteristics of the invention plant to nontransgenic plants that either have or have not been exposed to infection. Exemplary physical characteristics to observe include an increase in population of plants that have the ability to survive pathogen challenge, delayed lesion development, reduced lesion size, and the like. The term “disease” refers to a pathogen challenge caused any agent known to cause symptoms of infection in plants, including, but not limited to bacteria, nematodes, viruses, mycoplasmas, and fungi. In one embodiment, the pathogen is a bacterial pathogen, including, but not limited to, Pseudomonas. Exemplary organisms include, but are not limited to, Pseudomonas syringe pv. tomato (Pst) and Pseudomonas syringe pv. maculicola (Psm). The term “increased resistance to pathogens” or “increased resistance to disease” refers to a level of resistance that an invention transgenic plant has to plant pathogens above a defined reference level such as the level of resistance displayed by nontransgenic plants of the same species.

Thus, the increased resistance is measured relative to previously existing plants of the same species. In one embodiment, the resistance is substantially increased above the defined reference level greater than or equal to a 20% increase, greater than or equal to a 50% increase, greater than or equal to a 75% increase, or greater than or equal to a 95% increase and above. The phase “nontransgenic plant of the same species” means a plant of the same species that does not contain any heterologous transgenes, or has not been genomically targeted or modified. The levels of pathogen resistance can be determined using methods well known to one of skill in the art. These methods include bacterial resistance assays and fungal infection assays described in U.S. Pat. No. 5,530,187, herein incorporated by reference.

The term “exogenous,” as used herein, refers to a substance or molecule originating or produced outside of an organism. The term “exogenous gene” or “exogenous nucleic acid molecule,” as used herein, refers to a nucleic acid that codes for the expression of an RNA and/or protein that has been introduced (“transformed”) into a cell or a progenitor of the cell. An exogenous gene may be from a different species (and so a “heterologous” gene) or from the same species (and so a “homologous” gene), relative to the cell being transformed. A transformed cell may be referred to as a recombinant or genetically modified cell. An “endogenous” nucleic acid molecule, gene, or protein can represent the organism's own gene or protein as it is naturally produced by the organism.

The term “expression” refers to the process of converting genetic information of a polynucleotide into RNA through transcription, which is catalyzed by an enzyme, RNA polymerase. The term “expression” also refers to the process of converting genetic information into protein, through translation of mRNA on ribosomes. Expression can be, for example, constitutive or regulated, such as, by an inducible promoter (e.g., lac operon, which can be triggered by Isopropyl β-D-1-thiogalactopyranoside (IPTG)). Up-regulation or overexpression refers to regulation that increases the production of expression products (mRNA, polypeptide or both) relative to basal or native states, while inhibition or down-regulation refers to regulation that decreases production of expression products (mRNA, polypeptide or both) relative to basal or native states.

As used herein, the term “fermentation” refers to the enzymatic and/or anaerobic breakdown of organic substances by microorganisms to produce simpler organic compounds such as alcohols. In the case of ethanol fermentation, pyruvate is decarboxylated to yield acetaldehyde which is reduced by alcohol dehydrogenase to form ethanol. While fermentation may occur under anaerobic conditions, it is not intended that the term be solely limited to strict anaerobic conditions, as fermentation may also occur under aerobic (e.g., in the presence of oxygen) or microaerobic conditions.

The term “gene,” as used herein, refers to a segment of nucleic acid that encodes an individual protein or RNA and can include both exons and introns together with associated regulatory regions such as promoters, operators, terminators, 5′ untranslated regions, 3′ untranslated regions, and the like.

The term “homolog” means a gene related to a second gene by descent from a common ancestral DNA sequence, therefore, the corresponding polynucleotide/polypeptide has a certain degree of homology, that is to say sequence identity (preferably at least 40%, more preferably at least 60%, even more preferably at least 65%, particularly preferred at least 66%, 68%, 70%, 75%, 80%, 86%, 88%, 90%, 92%, 95%, 97% or 99%). “ANGUSTIFOLIA homolog” furthermore means that the function is equivalent to the function of the ANGUSTIFOLIA (AtAN) gene.

The term “increased size” or “increased mass” means an increase in size or mass of a treated plant of at least 5%, of at least 10%, or of at least 15% when compared to a corresponding untreated plant.

The terms “modulation”, “modulating” and “regulating” may be used interchangeably herein. In one embodiment, the term “modulation” refers to an increase and/or induction and/or promotion and/or activation. In an alternative embodiment, the term “modulation” refers to a decrease and/or reduction and/or inhibition.

As used herein, the term “nucleic acid” has its general meaning in the art and refers to refers to a coding or non coding nucleic sequence. Nucleic acids include DNA (deoxyribonucleic acid) and RNA (ribonucleic acid) nucleic acids. Examples of nucleic acid thus include but are not limited to DNA, mRNA, tRNA, rRNA, tmRNA, miRNA, piRNA, snoRNA, and snRNA. Nucleic acids thus encompass coding and non coding region of a genome (i.e. nuclear or mitochondrial).

A “nucleic acid inhibitor” is a nucleic acid that can reduce or prevent expression or activity of a target gene. For example, an inhibitor of expression of ANGUSTIFOLIA gene can reduce or eliminate transcription and/or translation of the ANGUSTIFOLIA gene product, thus reducing ANGUSTIFOLIA gene protein expression.

The term “operably linked” refers to positioning of a regulatory region and a sequence to be transcribed in a nucleic acid so as to influence transcription or translation of such a sequence. For example, to bring a coding sequence under the control of a regulatory region, the translation initiation site of the translational reading frame of the polypeptide is typically positioned between one and about fifty nucleotides downstream of the promoter. A regulatory region can, however, be positioned as much as about 5,000 nucleotides upstream of the translation initiation site or about 2,000 nucleotides upstream of the transcription start site. A regulatory region typically comprises at least a core (basal) promoter.

The term “regulatory region” refers to a nucleic acid having nucleotide sequences that influence transcription or translation initiation and rate and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns and combinations thereof.

A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene (Fromm et al., The Plant Cell 1:977-984 (1989)). The choice of regulatory regions to be included depends upon several factors, including, but not limited to, efficiency, selectability, inducibility, desired expression level and cell- or tissue-preferential expression. It is a routine matter for one of skill in the art to modulate the expression of a coding sequence by appropriately selecting and positioning regulatory regions relative to the coding sequence.

A “vector” is a carrier of genetic information, such as a plasmid, phage or cosmid, into which another DNA segment may be inserted so as to transport or deliver the inserted segment. In some embodiments, a vector is capable of replication when associated with the proper control elements. In other embodiments a vector is incorporated into a target genome and may replicate together with the genome. Suitable vector backbones include, for example, those routinely used in the art such as plasmids, viruses, artificial chromosomes, BACs, YACs or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses and retroviruses. Numerous vectors and expression systems are commercially available from such corporations as Novagen (Madison, Wis.), Clontech (Mountain View, Calif.), Stratagene (La Jolla, Calif.) and Invitrogen/Life Technologies (Carlsbad, Calif.).

As used herein, “vegetative growth period” refers to the time between germination and flowering (also known as the vegetative phase of plant development). During the vegetative phase, plants carry out photosynthesis and accumulate resources that will be needed for flowering and reproduction. Different types of plants show different growth habits. Flowering usually marks the end of vegetative growth period, therefore, delaying flowering may improve biomass accumulation in a plant.

As used herein, the term “extending vegetative plant growth” refers to extending the growth period of plant tissues between germination and flowering. In some embodiments, the term “extending vegetative plant growth” refers to an increase in root growth. In a specific embodiment, the root growth of a plant comprising an inactivated ANGUSTIFOLIA gene is increased by at least 5%, by at least 10%, or by at least 15% when compared to a corresponding control plant. In another embodiment, the term “extending vegetative plant growth” means an increase in shoot growth. More preferably, the shoot growth of a plant comprising an inactivated ANGUSTIFOLIA gene is increased by at least 5%, by at least 10%, or by at least 15% when compared to a corresponding control plant.

### General Description

The inventors of the present disclosure have determined that the ANGUSTIFOLIA (AtAN)/CtBP gene (AT1G01510, Genbank gene ID: 839401, AtAN Protein sequence accession number: O23702) is at the center of the carbon allocation between the Shikimate Pathway and the Pyruvate biosynthesis pathway underlying the observed salicylic acid (SA) and Jasmonic acid (JA)/ethylene (ET) antagonism. The proposed model of carbon allocation is based on the observation that phosphoenol pyruvate (PEP) is a shared precursor for pyruvate biosynthesis (subsequently leads to JA and ET biosynthesis) in the glycolysis pathway and 3-Deoxy-D-arabinoheptulosonate 7-phosphate (DAHP) biosynthesis in the shikimate pathway which leads to chorismate, SA and secondary cell wall biosynthesis. As such, regulation of the PEP shunt at this irreversible junction offers a tractable explanation for the antagonism as well as tradeoff between growth and immunity.

Disclosed herein are methods of controlling carbon flow between the Shikimate Pathway and the Pyruvate/Malate biosynthesis pathway in a plant by modulating the ANGUSTIFOLIA gene in said plant. Also disclosed herein are transgenic plants wherein the ANGUSTIFOLIA gene is modulated.

### Plants

The methods and compositions of the present disclosure can be used over a broad range of plant species, including species from the dicot genera Acer, Afzelia, Arabidopsis, Betula, Brassica, Eucalyptus, Fagus, Fraxinus, Glycine, Gossypium, Jatropha, Juglans, Linum, Lycopersicon, Medicago, Micropus, Populus, Prunus, Quercus, Salix, Solanum, Tectona and Trifolium; and the monocot genera Agrostis, Avena, Festuca, Hordeum, Lemna, Lolium, Milium, Miscanthus oryza, Panicum, Pennisetum, Phalaris, Phleum, Poa, Saccharum, Secale, Sorghum, Triticum, Zea and Zoysia; and the gymnosperm genera Abies, Picea and Pinus. In some embodiments, a plant is a member of the species Festuca arundinacea, Miscanthus hybrid (Miscanthus×giganteus), Miscanthus sinensis, Miscanthus sacchariflorus, Panicum virgatum, Pennisetum purpureum, Phalaris arundinacea, Populus spp including but not limited to balsamifera, deltoides, tremuloides, tremula, alba and maximowiczii, Saccharum spp., Secale cereale, Sorghum almum, Sorghum halcapense or Sorghum vulgare. In certain embodiments, the polynucleotides and vectors described herein can be used to transform a number of monocotyledonous and dicotyledonous plants and plant cell systems, wherein such plants are hybrids of different species.

### Inactivation of the ANGUSTIFOLIA Gene

The present inventors have discovered that inactivation of the ANGUSTIFOLIA gene in a plant results in increased disease resistance, extended vegetative growth period and delayed flowering time in said plant, which are desirable qualities to improve plant biomass.

In some embodiments, the ANGUSTIFOLIA gene is inactivated in a plant using targeted genome editing techniques. Targeted genome editing (also known as genome engineering) has emerged as an alternative to classical plant breeding and transgenic (Genetically Modified Organism—GMO) methods to improve crop plants. Available methods for targeted genome editing include the CRISPR/Cas system, zinc finger nucleases (ZFNs), and TAIL effector nucleases (TALENs). ZFNs are reviewed in Carroll, D. (Genetics, 188.4 (2011): 773-782), and TALENs are reviewed in Zhang et al. (Plant Physiology, 161.1 (2013): 20-27), which are incorporated herein in their entirety.

CRISPR/Cas system is a method based on the bacterial type II CRISPR (clustered regularly interspaced short palindromic repeats)/Cas (CRISPR-associated) immune system. The CRISPR/Cas system allows targeted cleavage of genomic DNA guided by a customizable small noncoding RNA, resulting in gene modifications by both non-homologous end joining (NHEJ) and homology-directed repair (HDR) mechanisms. CRISPR-Cas and similar gene targeting systems are well known in the art with reagents and protocols readily available. Exemplary genome editing protocols are described in Jennifer Doudna, and Prashant “CRISPR-Cas: A Laboratory Manual” (2016) (CSHL Press, ISBN: 978-1-621821-30-4) and Ran, F. Ann, et al. (Nature Protocols (2013), 8 (11): 2281-2308). Belhaj et al. (Plant Methods, 2013, 9:39) summarizes and discusses applications of the CRISPR/Cas technology in plants and is incorporated herein in its entirety.

In some embodiments, the inactivation of the ANGUSTIFOLIA gene is achieved by nucleic acid inhibitors of expression of the ANGUSTIFOLIA gene.

A number of nucleic acid based methods, including antisense RNA, ribozyme directed RNA cleavage, post-transcriptional gene silencing (PTGS), e.g., RNA interference (RNAi), microRNA and artificial microRNA and transcriptional gene silencing (TGS) can be used to inhibit ANGUSTIFOLIA gene expression in plants. Suitable inhibitors include full-length nucleic acids of allelic variants of ANGUSTIFOLIA gene, or fragments of such full-length nucleic acids. In some embodiments, a complement of the full-length nucleic acid or a fragment thereof can be used. Typically, a fragment is at least 10 nucleotides, e.g., at least 12, 13, 14, 15, 16, 17, 18, 19, 20, 21, 22, 23, 24, 25, 26, 27, 30, 35, 40, 50, 80, 100, 200, 500 nucleotides or more. Generally, higher homology can be used to compensate for the use of a shorter sequence.

Antisense technology is one well-known method. In this method, a nucleic acid fragment from a gene to be repressed is cloned and operably linked to a heterologous regulatory region and a transcription termination sequence so that the antisense strand of RNA is transcribed. The recombinant vector is then transformed into plants, as described below and the antisense strand of RNA is produced. The nucleic acid fragment needs not be the entire sequence of the gene to be repressed, but typically is substantially complementary to at least a portion of the sense strand of the gene to be repressed. By “substantially complementary” it is meant that the nucleic acid fragment is capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, 8%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization.

In another method, a nucleic acid can be transcribed into a ribozyme or catalytic RNA, which affects expression of an mRNA. See, U.S. Pat. No. 6,423,885. Ribozymes can be designed to specifically pair with a target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes cleave mRNAs at locations dictated by flanking regions that form complementary base pairs with the target mRNA. See, for example, U.S. Pat. No. 5,254,678; Perriman et al., PNAS 92(13):6175-6179 (1995); de Feyter and Gaudron, Methods in Molecular Biology, Vol. 74, Chapter 43, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence or a fragment thereof, of the polypeptide of interest. The length of the sequence that is similar or identical to the sense coding sequence can be from 10 nucleotides to 500 nucleotides, from 15 nucleotides to 300 nucleotides, from 20 nucleotides to 100 nucleotides or from 25 nucleotides to 100 nucleotides. The other strand of the stem portion of a double stranded RNA comprises a sequence that is similar or identical to the antisense strand or a fragment thereof, of the coding sequence of the polypeptide of interest and can have a length that is shorter, the same as or longer than the corresponding length of the sense sequence. In some cases, one strand of the stem portion of a double stranded RNA comprises a sequence that is similar or identical to the 3′ or 5′ untranslated region or a fragment thereof, of the mRNA encoding the polypeptide of interest and the other strand of the stem portion of the double stranded RNA comprises a sequence that is similar or identical to the sequence that is complementary to the 3′ or 5′ untranslated region, respectively or a fragment thereof, of the mRNA encoding the polypeptide of interest. In other embodiments, one strand of the stem portion of a double stranded RNA comprises a sequence that is similar or identical to the sequence of an intron or a fragment thereof in the pre-mRNA encoding the polypeptide of interest and the other strand of the stem portion comprises a sequence that is similar or identical to the sequence that is complementary to the sequence of the intron or fragment thereof in the pre-mRNA.

A construct including a sequence that is operably linked to a heterologous regulatory region and a transcription termination sequence and that is transcribed into an RNA that can form a double stranded RNA, can be transformed into plants as described below. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965; 20030175783, 20040214330 and 20030180945.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA or an intron in a pre-mRNA encoding a polypeptide of interest or a fragment of such sequences. In some embodiments, the sense or anti sense sequence is identical or complementary to a sequence of the regulatory region that drives transcription of the gene encoding a polypeptide of interest. In each case, the sense sequence is the sequence that is complementary to the antisense sequence.

A nucleic acid having at least one strand that is a template for one or more sense and/or antisense sequences can be operably linked to a regulatory region to drive transcription of an RNA molecule containing the sense and/or anti sense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a P-DNA such that the left and right border-like sequences of the P-DNA are on either side of the nucleic acid.

In some embodiments, a suitable nucleic acid inhibitor can be a nucleic acid analog. Nucleic acid analogs can be modified at the base moiety, sugar moiety or phosphate backbone to improve, for example, stability, hybridization or solubility of the nucleic acid. Modifications at the base moiety include deoxyuridine for deoxythymidine and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite or an alkyl phosphotriester backbone.

### Expression Vectors for Modulating the Activity of ANGUSTIFOLIA Gene

The polynucleotides and expression vectors described herein can be used to increase or inhibit expression of the ANGUSTIFOLIA gene.

The vectors provided herein can include origins of replication, scaffold attachment regions (SARs) and/or markers. A marker gene can confer a selectable phenotype on a plant cell. For example, a marker can confer biocide resistance, such as resistance to an antibiotic (e.g., kanamycin, G418, bleomycin or hygromycin) or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin or Flag-tag (Kodak, New Haven, Conn.) sequences typically are expressed as a fusion with the encoded polypeptide. Such tags can be inserted anywhere within the polypeptide, including at either the carboxyl or amino terminus. As described herein, plant cells can be transformed with a recombinant nucleic acid construct to express a polypeptide of interest.

A variety of promoters are available for use, depending on the degree of expression desired. For example, a broadly expressing promoter promotes transcription in many, but not necessarily all, plant tissues. Non-limiting examples of broadly expressing promoters that can be included in the nucleic acid constructs provided herein include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter and ubiquitin promoters such as the maize ubiquitin-1 promoter.

Some suitable regulatory regions initiate transcription, only or predominantly, in certain cell types. For example, a promoter that is active predominantly in a reproductive tissue (e.g., fruit, ovule or inflorescence) can be used. Thus, as used herein a cell type- or tissue-preferential promoter is one that drives expression preferentially in the target tissue, but may also lead to some expression in other cell types or tissues as well.

Root-active and root-preferential promoters confer transcription in root tissue, e.g., root endodermis, root epidermis or root vascular tissues. Root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990) and the tobacco RD2 promoter.

Promoters active in photosynthetic tissue confer transcription in green tissues such as leaves and stems. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab IR promoter from rice (Loan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis SUC2 sucrose-H+ symporter promoter (Truernit et al., Planta, 196:564-570 (1995)) and thylakoid membrane protein promoters from spinach (psaD, psaF, psaE, PC, FNR, atpC, atpD, cab, rbcS).

Lignin biosynthesis promoters are promoters that drive transcription of nucleic acids encoding enzymes involved in lignin biosynthesis. Examples of lignin biosynthesis promoters include promoters of the switchgrass (Panieum virgatum), rice (Oryza saliva), corn (Zea mays) and wheat (Triticum aestivum) homologs of the Populus cinnamate 4-hydroxylase, caffeoyl-CoA O-methyltransferase and caffeic acid O-methyltransferase genes. Also suitable are promoters of Arabidopsis genes encoding phenylalanin ammonia lyase (genomic locus At3g10340), trans-cinnamate 4-hydroxylase (genomic locus At2g30490), 4-coumarate:CoA ligase (genomic locus At1g51680), hydroxycinnamoyl-CoA:shikimate/quinate hydroxycinnamoyltransferase (genomic locus At5g48930), p-coumarate 3-hydroxylase (genomic locus At2g40890), caffeoyl-CoA 3-O-methyltransferase (genomic locus At4g34050), cinnamoyl CoA reductase (genomic locus At1g15950), ferulate 5-hydroxylase (genomic locus At4g36220), caffeic acid O-methyltransferase (genomic locus At5g54160) and cinnamyl alcohol dehydrogenase (genomic locus At4g34230).

Useful promoters also include cell wall related promoters, such as cellulose biosynthesis promoters. Cellulose biosynthesis promoters are promoters that drive transcription of nucleic acids encoding enzymes involved in cellulose biosynthesis. Examples of cellulose biosynthesis promoters include the promoter of the rice cellulose synthase gene (genomic locus Os08g25710), the promoter of the rice cellulose synthase gene (genomic locus Os08g06380) and the promoter of the rice cellulose synthase-like A2 gene (genomic locus Os10g26630).

Examples of promoters that have high or preferential activity in vascular bundles include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)) and the rice tungro bacilliform virus (RTBV) promoter (Dai et al., PNAS, 101(2):687-692 (2004)). Promoters having preferential activity in the phloem region (e.g., primary phloem cells, companion cells and sieve cells), the xylem region (e.g., tracheids and vessels), the bundle sheath layer and/or the endodermis are also considered vascular tissue promoters. Promoters that have preferential activity in the pith, cortex, epidermis and/or in the vascular bundles or vascular layers of the stem are considered stem promoters. In some cases, the activity of stem promoters can also be induced by stress like drought.

Inducible promoters confer transcription in response to external stimuli such as chemical agents or environmental stimuli. For example, inducible promoters can confer transcription in response to hormones such as gibberellic acid or ethylene or in response to light, nitrogen, shade or drought.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

A 5′ untranslated region (UTR) can be included in nucleic acid constructs described herein. A 5′ UTR is transcribed, but is not translated and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a vector, e.g., introns, enhancers, upstream activation regions, transcription terminators and inducible elements. Thus, for example, more than one regulatory region can be operably linked to the sequence of a polynucleotide encoding a Gene Y homolog or other lignin-modulating polypeptide. Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Techniques for introducing nucleic acids (inhibitors and expression vectors) into monocotyledonous and dicotyledonous plants are known in the art and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or tissue culture is used as the recipient tissue for transformation, plants can be regenerated from transformed cultures if desired, by techniques known to those skilled in the art. See, e.g., Niu et al., Plant Cell Rep. V19:304-310 (2000); Chang and Yang, Bot. Bull. Acad. Sin., V37:35-40 (1996) and Han et al., Biotechnology in Agriculture and Forestry, V44:291 (ed. by Y. P. S. Bajaj), Springer-Vernag, (1999).

### Transgenic Plants/Plant Species/Plant Cells

Also disclosed herein are plants and plant cells genetically modified by introduction of the disclosed nucleic acid inhibitors, CRISPR constructs and expression vectors.

A plant or plant cell used in methods of the invention contains a recombinant nucleic acid construct as described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Typically, transgenic plant cells used in methods described herein constitute part or all of a whole plant. Such plants can be grown in a manner suitable for the species under consideration, either in a growth chamber, a greenhouse or in a field. Transgenic plants can be bred as desired for a particular purpose, e.g., to introduce a recombinant nucleic acid into other lines, to transfer a recombinant nucleic acid to other species or for further selection of other desirable traits. Progeny includes descendants of a particular plant or plant line provided the progeny inherits the transgene. Progeny of a plant include seeds formed on F1, F2, F3, F4, F5, F6 and subsequent generation plants or seeds formed on BC1, BC2, BC3 and subsequent generation plants or seeds formed on F1BC1, F1BC2, F1BC3 and subsequent generation plants. Seeds produced by a transgenic plant can be grown and then selfed (or outcrossed and selfed) to obtain seeds homozygous for the nucleic acid construct. Alternatively, transgenic plants can be propagated vegetatively for those species amenable to such techniques.

Transgenic plant cells growing in suspension culture or tissue or organ culture can be useful for extraction of polypeptides or compounds of interest, e.g., lignin monomers or compounds in a lignin biosynthetic pathway. For the purposes of this invention, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be any of various mineral salt media, e.g., Murashige and Skoog (MS) medium containing agar and a suitable concentration of an auxin, e.g., 2,4-dichlorophenoxyacetic acid (2,4-D) and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days or about 1-3 days. The use of transient assays is particularly convenient for rapid analysis in different species or to confirm expression of a heterologous ANGUSTIFOLIA gene allelic variant whose expression has not previously been confirmed in particular recipient cells.

Initial and immediate application of the expression of ANGUSTIFOLIA gene allelic variants can be made in the bioenergy crops Populus and switchgrass, but the application can be extended to other bioenergy crops such as corn, other sources of lignocellulosic biomass and other model plants e.g., Salix, Miscanthus, rice and Medicago.

For example, the polynucleotides and vectors described herein can be used to transform a number of monocotyledonous and dicotyledonous plants and plant cell systems, including alfalfa, ash, beech, birch, canola, cherry, clover, cotton, cottonseed, eucalyptus, flax, jatropha, mahogany, maple, mustard, oak, poplar, oilseed rape, rapeseed (high erucic acid and canola), red clover, teak, tomato, walnut and willow, as well as monocots such as barley, bluegrass, canarygrass, corn, fescue, field corn, millet, miscanthus, oat, rice, rye, ryegrass, sorghum, sudangrass, sugarcane, sweet corn, switchgrass, turf grasses, timothy and wheat. Gymnosperms such as fir, pine and spruce can also be suitable.

The methods and compositions can be used over a broad range of plant species, including species from the dicot genera Acer, Afzelia, Arabidopsis, Betula, Brassica, Eucalyptus, Fagus, Fraxinus, Glycine, Gossypium, Jatropha, Juglans, Limum, Lycopersicon, Medicago, Micropus, Populus, Prunus, Quercus, Salix, Solanum, Tectona and Trifolim; and the monocot genera Agrostis, Avena, Festuca, Hordeum, Lemna, Lolium, Milium, Miscanthus oryza, Panicum, Pennisetum, Phalaris, Phleum, Poa, Saccharum, Secale, Sorghum, Triticum, Zea and Zoysia; and the gymnosperm genera Abies, Picea and Pinus. In some embodiments, a plant is a member of the species Festuca arundinacea, Miscanthus hybrid (Miscanthus×giganteus), Miscanthus sinensis, Miscanthus sacchariflorus, Panicum virgatum, Pennisetum purpureum, Phalaris arundinacea, Populus spp including but not limited to balsamifera, deltoides, tremuloides, tremula, alba and maximowiczii, Saccharum spp., Secale cereale, Sorghum almum, Sorghum halcapense or Sorghum vulgare. In certain embodiments, the polynucleotides and vectors described herein can be used to transform a number of monocotyledonous and dicotyledonous plants and plant cell systems, wherein such plants are hybrids of different species.

### Methods of Modulating Plant Phenotypes Using Expression Vector Modulators of the ANGUSTIFOLIA Gene

This disclosure provides methods of altering growth period, flowering time, disease resistance, lignin synthesis and sugar release in a plant, comprising introducing into a plant cell an exogenous nucleic acid with a regulatory region operably linked to a nucleic acid encoding a ANGUSTIFOLIA gene allelic variant, where a tissue of a plant produced from the plant cell has an altered cell wall compared to the cell wall in tissue of a control plant that does not comprise the nucleic acid.

In one embodiment, the exogenous nucleic acid is an expression vector encoding the polypeptide of a ANGUSTIFOLIA gene allelic variant that leads to low, inhibited or decreased lignin synthesis. An example of such an expression vector is an expression vector comprising the ANGUSTIFOLIA gene allelic variant encoding SEQ ID NO: 1. Expression of such a vector in a plant or plant cell would lead to a decrease in lignin synthesis in that plant or plant cell. This expression vector would be useful, for example, for increasing sugar release, that is, increasing glucose and/or xylose release, in a plant or plant cell in which the expression vector is introduced, relative to plants or plant cells which are not transformed by the vector. This expression vector would also be useful for decreasing lignification or lignin production in a plant or plant cell in which the expression vector is introduced.

In another embodiment, the exogenous nucleic acid is an expression vector encoding the polypeptide of a ANGUSTIFOLIA gene allelic variant that leads to delayed flowering, extended growth period, high or increased lignin synthesis. An example of such an expression vector is an expression vector comprising the ANGUSTIFOLIA gene allelic variant encoding SEQ ID NO: 1. This expression vector would be useful, for example, for increasing lignin synthesis in a plant or plant cell in which the expression vector is introduced, relative to plants or plant cells which are not transformed by the vector.

In one example, the coding sequence of an ANGUSTIFOLIA gene allelic variant is amplified from either genomic DNA or cDNA by PCR. The DNA fragments are then subcloned into an expression construct. In this example, a construct is made by first digesting pSAT4A-DEST-n(1-174)EYFP-N1 (ABRC stock #CD3-1080) and pSAT5-DEST-c(175-end)EYFP-C1(B) (ABRC stock #CD3-1097) (Citovsky V. et al., J. Mol Biol 362:1120-1131 (2006)) with NdeI and BglII, then ligating the 1.1 kb fragment of the first construct and 4.4 kb fragment of the second one, followed by subcloning of the coding sequence of a ANGUSTIFOLIA gene allelic variant into the construct to create the expression vector.

### Methods of Use of Transgenic Plants

Disclosed herein are methods of using transgenic plants with reduced or inhibited expression or activity of the ANGUSTIFOLIA gene in a bioproduct conversion process, for instance, for producing a bioenergy product, a biomaterial, a biopharmaceutical and a biocosmetics. In some embodiments, the bioenergy product is selected from the group consisting of ethanol, biodiesel, and biogas. In some embodiments, the biomaterial is selected from the group consisting of bioplastics, biofoams, biorubber, biocomposites, and biofibres. In some embodiments, the biopharmaceutical product is anatural source medicinal compound. In some embodiments, the biocosmetics is selected from the group consisting of soaps, body creams, shampoos, lotions, and herbal extracts made using the transgenic plants described in this application.

Further disclosed herein are improved methods of producing biofuel from cellulosic biomass, by using plants with reduced or inhibited expression or activity of the ANGUSTIFOLIA gene in biofuel production processes. Methods of pretreatment and saccharification of biomass to fermentable sugars, followed by fermentation of the sugars to ethanol, are known in the art. Ethanol fermentation process, also called alcoholic fermentation process, is a biological process which converts sugars such as glucose, fructose, and sucrose into cellular energy, producing ethanol and carbon dioxide as a side-effect. In some embodiments this conversion takes places in the absence of oxygen.

The overall chemical formula for ethanol/alcohol fermentation is:

C₆H₁₂O₆→2C₂H₅OH+2CO₂

Additionally disclosed are methods of producing paper and pulp, by using plants with reduced or inhibited expression or activity of the ANGUSTIFOLIA gene in paper or pulp production processes, as known in the art. Further disclosed are pulp and paper products produced by this method, using plants with increased expression of the ANGUSTIFOLIA (ATAN)/CTBP gene.

A specific aspect of the disclosure provides a method of improving biomass yield and lengthening the vegetative growth period in a plant inactivating the ANGUSTIFOLIA/CtBP (ANGUSTIFOLIA (ATAN)/CTBP) gene.

Another embodiment provides a plant, a plant cell or a plant tissue with improved biomass production comprising a loss of function mutation in ANGUSTIFOLIA(AtAN)/CtBP gene.

### Articles of Manufacture

The materials and methods described herein are useful for modifying biomass characteristics. “Biomass” refers to any cellulosic or lignocellulosic raw material and includes materials containing cellulose, and optionally further containing hemicellulose, lignin, starch, oligosaccharides and/or monosaccharides. The term “cellulosic” refers to a composition containing cellulose. The term “lignocellulosic” refers to a composition containing both lignin and cellulose. According to the invention, biomass may be derived from a single source, or biomass can contain a mixture derived from more than one source; for example, biomass can contain a mixture of corn cobs and corn stover, or a mixture of grass and leaves. Examples of biomass include, but are not limited to, tree crops such as Populus, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from processing of grains, trees, branches, roots, leaves, wood chips, sawdust, shrubs and bushes, vegetables, and fruits.

Lignin itself, which can be gathered from transgenic plants provided herein, can be converted into valuable fuel additives. Lignin can be recovered from any bioethanol production process using agricultural materials such as straw, corn stalks and switchgrass engineered to have increased lignin content. Lignin can be combusted to provide heat and/or power for the ethanol process; however, increasing the value of the lignin by converting it to higher value fuel additives can significantly enhance the competitiveness of bioethanol technology. Lignins removed from wood pulp as sulphates can be used as dust suppression agents for roads, as dispersants in high performance cement applications, water treatment formulations and textile dyes or as raw materials for several chemicals, such as vanillin, DMSA, ethanol, torula yeast, xylitol sugar and humic acid.

The invention also relates to the use of the pulp obtained from the disclosed genetically modified plants in the production of cellulose-based products, for example, in the paper industry, or for the production of cardboard. Pulp, produced using plants which have been genetically modified to have decreased expression of the ANGUSTIFOLIA gene and therefore also have extended vegetative growth period, can be used as a building material and in particular as output material for pressed chipboard, fiberboard of medium density, or as filler material.

### EXAMPLES

### Example 1

### Materials and Methods

### Plant Materials and Growth Conditions

Arabidopsis plants used in this study were grown in a growth chamber with 12 h light at 23° C./12 h dark at 20° C. with 60% relative humidity. The T-DNA insertional mutant an-1 (Gachomo et al., 2013, Bmc Plant Biology, 13, 1) was obtained from the ABRC (Arabidopsis Biological Resource Center, The Ohio State University). For generating an-1 355:AN transgenic plants, full-length of AN was cloned into pENTR vector and then subcloned into binary vector pGWB515 for transformation into an-1 mutant background.

### Protoplast Isolation and Transfection

Arabidopsis mesophyll protoplasts were isolated and transfected as described previously (Yoo et al., Nat Protoc., 2, 1565-1572 (2007)). Protoplasts were isolated from fully expanded leaves from 3-4-week-old Col-0 plants. The concentration of isolated protoplasts was adjusted to 2×10⁵ ml⁻¹. Plasmid constructs were then transfected into protoplasts using PEG-calcium transfection method (Yoo et al., Nat Protoc., 2, 1565-1572 (2007)). For subcellular localization analysis, 8 μg of AN-YFP plasmid DNA and 2 μg of CFP-SHY2 or Golgi-mCherry plasmid DNA were co-transfected into 100 μl of protoplasts. For co-localization analysis, 5 μg of AN-YFP plasmid DNA was co-transfected with 5 μg of either mCherry-TDP1 or mCherry-TDP1Δ1-122 plasmid DNA into 100 μl of protoplasts. For cell fraction analysis, 20 μg of AN-Myc plasmid DNA was transfected into 200 μl of protoplasts. Five reactions were combined to obtain one ml of protoplasts for subsequent cell fraction and western blotting analyses. To determine the effect of TDP1 on AN nuclear accumulation, 15 μg of HA-TDP1 and 5 μg of AN-Myc plasmid DNA were co-transfected into 200 μl of protoplasts. To balance the protein level of AN-Myc, the AN-Myc only reaction was performed by co-transfecting 15 μg of blank vector and 5 μg of AN-Myc plasmid DNA into 200 μl of protoplasts. For transcriptional activity assays, a total 10 μg plasmid DNA including reporter, effector, and/or transactivator plasmid DNA were co-transfected into 100 μl of protoplasts. To compare the effect of different effector constructs, the same amount of effect constructs was used. For single protein ChIP, 20 μg of plasmid DNA containing AN-Myc or HA-TDP1 was transfected into 200 μl of protoplasts. After overnight incubation, protoplasts were crosslinked and then used for ChIP. For AN and TDP1 co-expression, 10 μg of plasmid expressing AN-Myc and 10 μg of plasmid expressing HA-TDP1 were co-transfected into 200 μl of protoplasts. To balance the protein level of AN-Myc, the AN-Myc only reaction was performed by co-transfecting 10 μg of blank vector and 10 μg of AN-Myc construct into 200 μl of protoplasts.

### μChIP-qPCR

μChIP was performed as previously described (Xie et al., The Plant Cell, tpc. 00168.02018 (2018)). AN-Myc and HA-AtTDP1 plasmid DNAs were transiently expressed in protoplasts. After 14 h incubation at room temperature, approximately 40,000 transfected protoplasts were used for μChIP. Cells were crosslinked by 1% formaldehyde in W5 Solution (154 mM NaCl, 125 mM CaCl₂, 5 mM KCl, 2 mM MES pH 5.7) for 8 min at room temperature with gentle rotation. Formaldehyde was subsequently quenched by adding 1.25 M Glycine (Sigma-Aldrich) to final concentration of 125 mM and incubating for 5 min at room temperature with gentle rotation. After two times washing with W5 Solution, cells were collected by centrifugation (2000 rpm for 10 min, 4° C.) and lysed in 50 μl of Lysis Buffer (50 mM Tris-HCl pH 8.0, 100 mM NaCl, 10 mM EDTA pH 8.0, 1% SDS, 1 mM PMSF, and protease inhibitor (Sigma-Aldrich)) with intermittent vortexing for 20 min. The concentration of SDS was then diluted to less than 0.1% by adding 800 μl of ChIP Dilution Buffer (0.01% SDS, 1.1% Triton X-100, 1.2 mM EDTA, 16.7 mM Tris-HCl pH 8.0, 167 mM NaCl, 1 mM PMSF, and protease inhibitor (Sigma-Aldrich)). After dilution, cell lysis was sonicated for 150 s with 0.7 s ‘On’ and 1.3 s ‘Off’ pulses at 20% power amplitude using the Branson 450 Digital sonifier machine to achieve chromatin fragments of 150-600 bp (Adli and Bernstein, Nature Protocols, 6, 1656-1668 (2011)). The sonicated cell lysis was added by an additional 150 μl of ChIP Dilution Buffer and centrifuged at 10,000 g for 10 min at 4° C. to remove cellular debris. After centrifugation, the supernatant was aliquoted into three 1.5 ml tubes: 25 μl for Input sample, 450 μl for IgG control, 450 μl for ChIP with antibody. Additional ChIP Dilution Buffer was then added: 75 μl for Input sample, 450 μl for IgG control, 450 μl for ChIP with antibody. Anti-Myc (Sigma) and anti-HA antibodies (Sigma) were used to immuneprecipitate AN-Myc and HA-AtTDP1, respectively. ChIPed DNA and the input DNA were then cleaned and concentrated using Qiagen MinElute PCR Purification Kit (Qiagen). qPCR was performed to quantify DNA enrichment. Three biological replicates were performed. Primers used for qPCR are listed in Table 1.

TABLE 1
Primers used in this study

| Name | Sequence (5′-3′) | Applications |
|---|---|---|
| AN CDS-F | CACCATGAGCAAGATCCGTTCGTCTG (SEQ ID NO: 3) | AN cloning |
| AN-CDS-R | TTAATCGATCCAACGTGTGATA (SEQ ID NO: 4) | |
| TDP1-pGBKT7-F | CACCATGGCTCACTCTCAGGTTGCTT (SEQ ID NO: 5) | TDP1 |
| TDP1-pGBKT7-R | ATGTCGACCTATCTTGGCCAGACTTGTCCA (SEQ ID NO: 6) | |
| P-MYB46-F | CACCAAAAGGAGTATAACATTTTCTT (SEQ ID NO: 7) | Promoter |
| P-MYB46-R | CTATCTTGGCCAGACTTGTCCA (SEQ ID NO: 8) | |
| P-WRKY33-F | CACCCAAACTCACTTCTAAATACTAA (SEQ ID NO: 9) | Promoter |
| P-WRKY33-R | CTGTATATTTGTTGGTTATGTC (SEQ ID NO: 10) | |
| AN qRT-F | GTCTTCCCCAAATCAGCTTG ((SEQ ID NO: 11) | qPCR |
| AN qRT-R | CCGAGAGGTCTTTCGCATAC (SEQ ID NO: 12) | |
| TDP1 qRT-F | TTACCTGGCCGTGGTAATGT (SEQ ID NO: 13) | qPCR |
| TDP1 qRT-R | GGTATCCAGGGACTGAAGCA (SEQ ID NO: 14) | |
| WRKY33 qRT-F | CGATTGTCAGCAGAGACGAA (SEQ ID NO: 15) | qPCR |
| WRKY33 qRT-R | TCCATCTGTAACCGTCGTCA (SEQ ID NO: 16) | |
| ACS6 qRT-F | GACGAGTTTATCCGCGAGAG (SEQ ID NO: 17) | qPCR |
| ACS6 qRT-R | ACACGCCATAGTTCGGTTTC (SEQ ID NO: 18) | |
| ACS2 qRT-F | CATGCTTGCTTCGATGTTGT (SEQ ID NO: 19) | qPCR |
| ACS2 qRT-R | CTTGATCCCCGTGGTAAAAA (SEQ ID NO: 20) | |
| ERF1A qRT-F | CGGTCAAGAAGGAGAAGACG (SEQ ID NO: 21) | qPCR |
| ERF1A qRT-R | CTTCGCCGGGTCTCTAATCT (SEQ ID NO: 22) | |
| UBQ qRT-F | GGTGCTAAGAAGAGGAAGAAT (SEQ ID NO: 23) | qPCR |
| UBQ qRT-R | CTCCTTCTTTCTGGTAAACGT (SEQ ID NO: 24) | |
| MYB46 ChIP-F | CCATGACCGATCAACTAACG (SEQ ID NO: 25) | ChIP-qPCR, |
| MYB46 ChIP-R | GAACCCTGGCTCTTTTTCAA (SEQ ID NO: 26) | |
| WRKY33 ChIP-F | TCATACGTGTCAGAACGAGACA (SEQ ID NO: 27) | ChIP-qPCR |
| WRKY33 ChIP-R | CAGACCTTGTGGCCTTGACT (SEQ ID NO: 28) | |
| ACS6 ChIP-F | ATGAAAAGAATTCCGGTCCA (SEQ ID NO: 29) | ChIP-qPCR |
| ACS6 ChIP-R | TTGGAAAAGAAATGAGACATCAA (SEQ ID NO: 30) | |
| ACS2 ChIP-F | AAATTCCCTTCCCAAATGGT (SEQ ID NO: 31) | ChIP-qPCR |
| ACS2 ChIP-R | ACAAGCGAACCAAGGAAAAA (SEQ ID NO: 32) | |
| ERF1A ChIP-F | CCAATCACAACATTGCTTCG (SEQ ID NO: 33) | ChIP-qPCR |
| ERF1A ChIP-R | AAACACGTGCGTTTTATCCA (SEQ ID NO: 34) | |
| ACTIN ChIP-F | CGTTTCGCTTTCCTTAGTGTTAGCT (SEQ ID NO: 35) | ChIP-qPCR |
| ACTIN ChIP-R | AGCGAACGGATCTAGAGACTCACCTTG (SEQ ID NO: 36) | |
| MYB58 ChIP-F | CGTCGAGAAATGTTGTGTGTG (SEQ ID NO: 37) | ChIP-PCR |
| MYB58 ChIP-R | TGGGTCCTATAACCCTGTAACAT (SEQ ID NO: 38) | |
| MYB103 ChIP-F | TTTATAAAATAATAGGTCAACCTCGAA (SEQ ID NO: 39) | ChIP-PCR |
| MYB103 ChIP-R | CATGTATTATCCACTGTTTTCCTCT (SEQ ID NO: 40) | |
| MYB63 ChIP-F | TGCATCGGTGTTAGAAGGAA (SEQ ID NO: 41) | ChIP-PCR |
| MYB63 ChIP-R | TTGTTGAGTGGGAAAAGGTTG (SEQ ID NO: 42) | |
| MYB55 ChIP-F | TCTACAATACTACCAAACAGAACCAAA (SEQ ID NO: 43) | ChIP-PCR |
| MYB55 ChIP-R | GAGAGGAGGATTTGGGGAAT (SEQ ID NO: 44) | |
| MYB20 ChIP-F | GATTGAGCTCATAGTCCCGTTT (SEQ ID NO: 45) | ChIP-PCR |
| MYB20 ChIP-R | TTTTCTTATTTCGTGTCACTTTGG (SEQ ID NO: 46) | |
| NAC073 ChIP-F | TTTGTTTGATCAGTCTTTGTCCA (SEQ ID NO: 47) | ChIP-PCR |
| NAC073 ChIP-R | TTGCTTGGGTTTTAAGTTTGG (SEQ ID NO: 48) | |
| WRKY53 ChIP-F | CACTCTGGCCCTATACTTCCTT (SEQ ID NO: 49) | ChIP-PCR |
| WRKY53 ChIP-R | TTGACCAAATGACCAAACCA (SEQ ID NO: 50) | |
| WRKY26 ChIP-F | ATTCAGCCGCCTTACACAAA (SEQ ID NO: 51) | ChIP-PCR |
| WRKY26 ChIP-R | TCCAAGGAAAAGCAAGCAAT (SEQ ID NO: 52) | |
| WRKY22 ChIP-F | ACAAACCGAACCGCTTTTTA (SEQ ID NO: 53) | ChIP-PCR |
| WRKY22 ChIP-R | AGAACAAACCGCTGCAAACT (SEQ ID NO: 54) | |
| WRKY40 ChIP-F | GCCGGCTATGCTATAACGAA (SEQ ID NO: 55) | ChIP-PCR |
| WRKY40 ChIP-R | TATGACGCTCTCCACGTTTG (SEQ ID NO: 56) | |

### Gene Expression Analysis

Total RNAs were extracted from one ml of transfected protoplasts using Plant RNA extraction kit (Sigma, St Louis, Mo.). On-column DNA digestion was performed as per the manufacturer recommendation. Two μg of total RNA was used for cDNA synthesis. qRT-PCR was then performed using cDNA as template and following the manual of Maxima SYBR Green qPCR Master Mixes (Thermo Scientific). Three replicates were performed for each sample. Gene expression was calculated by ΔΔC_{T} method using the expression of housekeeping gene (ACTIN) for template normalization.

### Electrophoretic Mobility Shift Assay

AN was cloned into the pGEX-6P-1 vector (GE Healthcare) with BamHI for GST fusion constructs. The constructs were transformed into E.coli strain BL21(DE3)pLysS (Invitrogen) for protein expression. GST fusion proteins were extracted and purified as previously described using Glutathione Sepharose 4B beads (GE Healthcare) (Xie et al., Nucleic Acids Res, 40, 4422-4431 (2012)). GST-AN proteins were eluted from beads by incubating with Elution Buffer (50 mM Tris-HCl pH 8.0, 10 mM reduced glutathione) at 4° C. for 30 min. For DNA probes, −447-bp to −300-bp promoter region of AtMYB46 was amplified by PCR from Col-0 genomic DNA, gel purified, and end labeled with biotin using DNA 3′ End Biotinylation Kit (Thermo Scientific) according to the manufacturer's manual. The DNA binding reaction included 0.25 nM Biotin-labeled probe, 0.4 μg of purified protein, 10 mM Tris-HCl (pH 7.5), 50 mM KCl, 1 mM DTT, 2.5% Glycerol, 5 mM MgCl2, 1 μg Poly (dI-dC), 0.05% NP-40. Reactions were incubated at room temperature for 20 min. The reaction mixtures were then resolved in 6% DNA retardation gel (Novex) by electrophoresis at 100 V for 1-2 h and transferred to Nylon membrane. Signals of biotin were detected using Chemiluminescent Nucleic Acid Detection Module (Thermo Scientific) as suggested by manufacturer.

### Transcriptional Activity Assay

The protoplast transfection-based transcriptional activity assay was performed according to the method described by Tiwari et al. (Tiwari et al., Plant Cell, 16, 533-543 (2004)). Reporter and effector constructs were co-transfected into protoplasts and incubated under the darkness for 18-20 h at room temperature. GUS activity assay was performed as described (Yoo et al., Nat Protoc, 2, 1565-1572 (2007)). GUS activity was measured using Fluoroskan microplate reader. To normalize GUS activity, 100 ng of 35S:luciferase plasmid was co-transfected for each reaction. Luciferase activity was measured using Promega Luciferase Assay System according to the manufacture manual. GUS activity in individual samples was normalized against luciferase activity (GUS/LUC). Three replicates were performed for statistical calculation.

### Subcellular Localization Analysis

AN-YFP construct was co-transfected with nuclear or Golgi markers into protoplasts to determine the subcellular localization of AN. For colocalization, paired constructs were co-transfected into protoplasts. After 14 h incubation under weak light at room temperature, protoplasts were collected and resuspended in cold W5 solution (2 mM MES pH 5.7, 154 mM NaCl, 125 mM CaCl₂, and 5 mM KCl) to subject to microscopy. Images were collected using a Zeiss LSM 710 confocal microscope, equipped with 458, 514 and 561 nm laser lines for excitation of CFP, YFP and mCherry, respectively. Images were processed using Zen software (Zeiss).

### Cell Fractionation and Protein Gel Blots

One ml of transfected protoplasts was incubated at room temperature for 14 h to express protein and then collected by centrifuge. The cytosolic and nuclear fractions were separated as previously described (Xie et al., The Plant Cell, tpc. 00168.02018 (2018)). Total protein isolation was extracted by incubating protoplasts in extraction buffer (20 mM Tris-HCl pH 7.4, 25% glycerol, 20 mM KCl, 2 mM EDTA, 2.5 mM MgCl₂, 250 mM sucrose, 1 mM DTT, and 1 mM PMSF) for 1 h at 4° C. After centrifugation at 1,500×g for 10 min at 4° C., the clear supernatant was taken as the cytosolic fraction (enriched using acetone precipitation). The pellet was washed twice with nuclei resuspension Triton buffer (20 mkt Tris-HCl pH 7.4, 25% glycerol, 2.5 mM MgCl₂, 0.2% Triton X-100) and once with nuclei resuspension buffer (20 mM Tris-HCl pH 7.4, 25% glycerol, 2.5 mM MgCl₂). Cytosolic and nuclear proteins were then separated by SDS/PAGE and transferred to polyvinylidene difluoride (PVDF) membrane (Bio-rad). Anti-Myc (Sigma, C3956), anti-HA (Sigma, H3663), anti-histone H3 (Abeam, ab1791), and anti-UGPase (Agrisera, AS05 086) were used as primary antibodies, Anti-Rabbit IgG peroxidase antibody (Sigma, A9169) was used as the secondary antibody for anti-Myc, anti-histone H3, and anti-UGPase. Anti-mouse IgG peroxidase antibody (Sigma, A9044) was used as the secondary antibody for anti-HA. Chemiluminescent signals were generated using the ECL Western Blotting Detection Reagents (GE Health) and detected with ChemiDoc XRS+ system (Bio-rad). To blot one membrane for multiple times, the imaged membrane was washed with strip buffer (Thermo Scientific) and subjected to the next blotting.

### Yeast Two-Hybrid Assay

pGADT7 and pGBKT7 plasmid pairs containing various genes were co-transformed into yeast Y2H Gold (Clonetech). SD-Leu-Trp plate was used to select yeasts containing both constructs. The resulting clones were diluted in 50 μl water and 5 μl was used for spot assay on SD-Leu-Trp-Ade-His plates. The interactions of AN and TDP1 activate the expression of Ade and His, which enables the growth of Y2H Gold cells in Ade and His minus plates. Three independent clones were tested for each plasmid pair.

### Lignin Staining

Lignin staining of root tissues was performed as previously described (Taylor-Teeples et al., Nature, 517, 571-575 (2015)). Seven-day-old seedlings growing on ½ MS plates were fixed in a 3:1 95% ethanol:glacial acetic solution for 10 min. Then samples were stained with 1% phloroglucinol solution in 50% HCl for 5 min. Seedlings were then mounted in 50% glycerol on slides and viewed using Zeiss light microscope. A total of 20 independent seedlings were observed for each genotype.

### Pathogen Infection

Detached 4-week-old Arabidopsis leaves were inoculated with B. cinerea as previously described (Ingle and Roden, Plant Circadian Networks: Methods and Protocols, 273-283(2014)). B. cinerea was grown on potato dextrose agar (Difco) plate at 25° C. in the dark for 10 days before collection of spores. The grey conidiospores were then harvested in sterile water and filtered through glass wool to remove mycelium. Four-week-old Arabidopsis leaves were detached from plants and transferred on 1% agar plate and inoculate with 5 μl (5×10⁵ spores/ml) of conidiospore suspension (two spots per leaf). Leaf lesions were pictured and measured every 24 h using Zeiss dissecting microscope. For each genotype, 20 leaves (40 spots) from independent plants were used for statistical analysis.

### Example 2

### ANGUSTIFOLIA (AtAN)/CtBP Gene, the Transcriptional Regulator of PEP Shunt Between Shikimate and Pyruvate Biosynthesis

While characterizing the molecular basis of a significant genome wide association (GWAS) correlation between an ANGUSTIFOLIA/CtBP and cell wall phenotypes in Populus, transcriptional changes in an Arabidopsis T-DNA insertion line with loss of function of the only ANGUSTIFOLIA (AtAN)/CtBP in the genome were characterized.

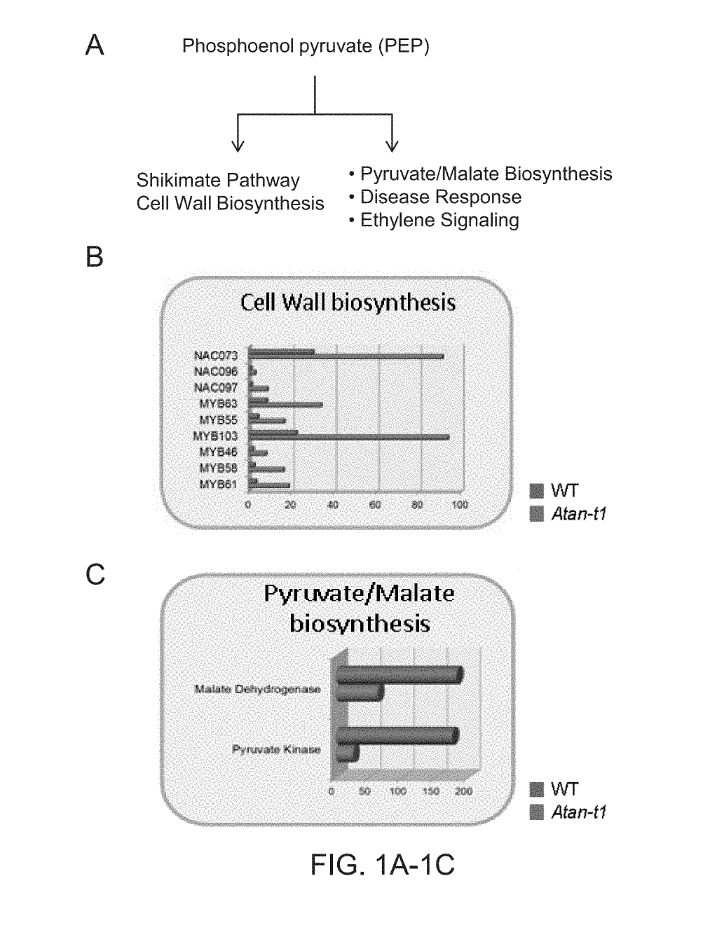

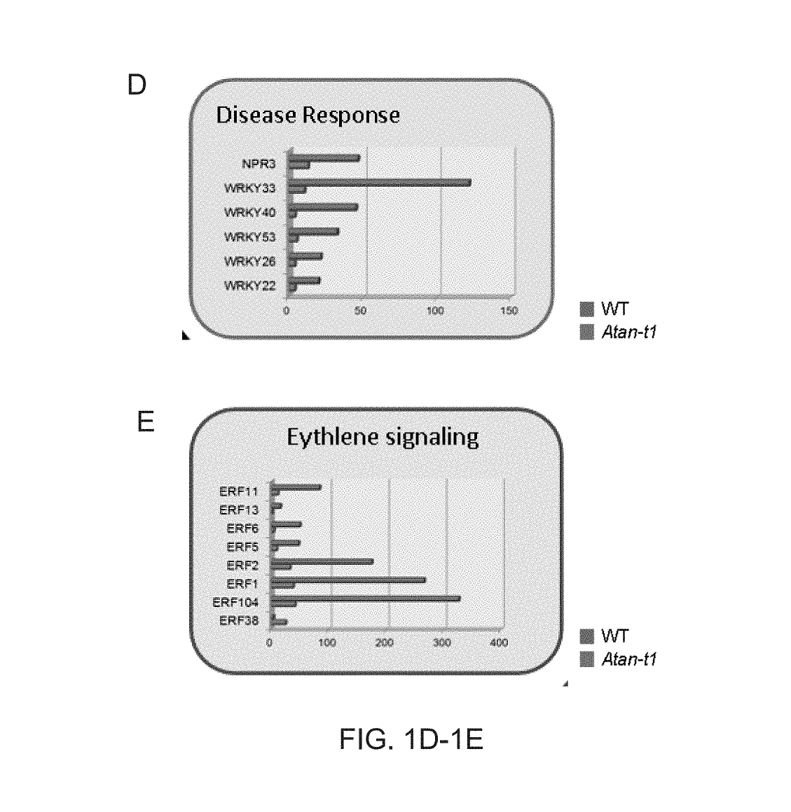

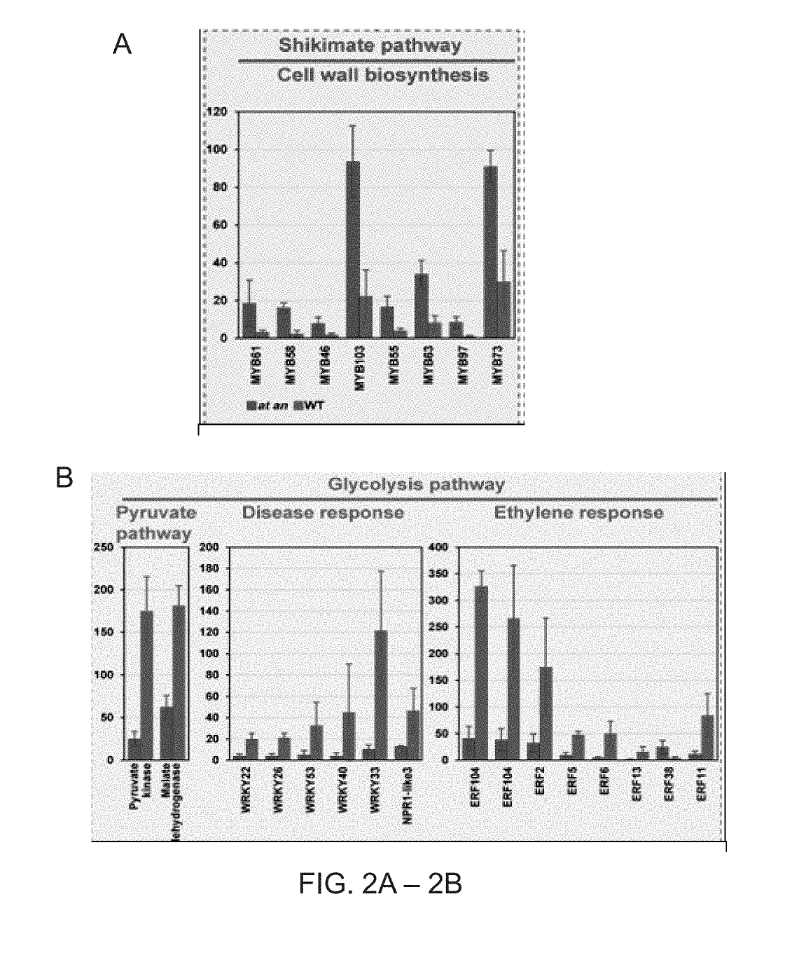

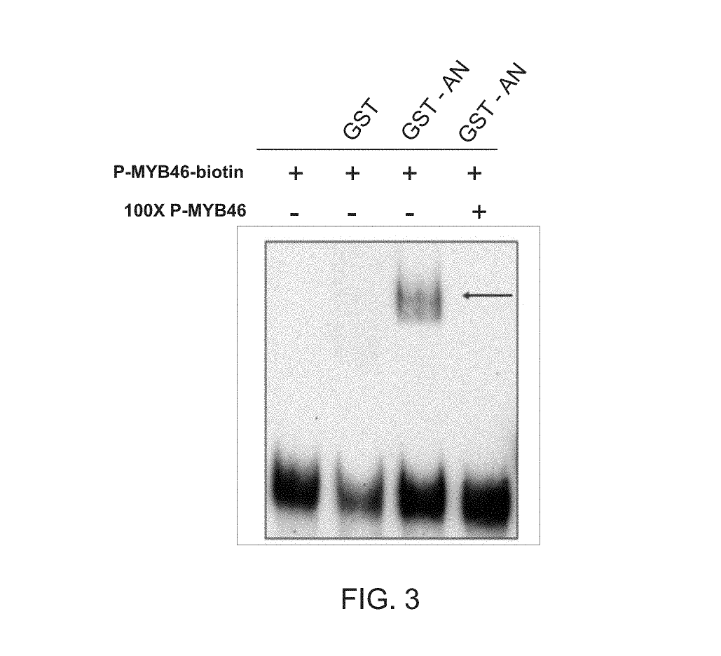

The expression of genes encoding Pyruvate kinase and malate dehydrogenase was significantly downregulated in the mutant lines compared to wild type. Since these two genes lie at the entry point of PEP into pyruvate biosynthesis, the responses of genes in the competing Shikimate Pathway were evaluated for evidence of increased PEP shunt in that direction. There was a significant up-regulation of genes associated with shikimate and cell wall biosynthesis in the mutant line. In fact, nine transcription factors with verified regulatory activity of secondary cell wall and phenyl propanoid pathways exhibited at least 2-fold upregulation in the mutant line (FIG. 1). Of these includes the master regulator MYB46. Beyond transcriptional regulators, virtually all major classes of cell wall biosynthesis also showed increased expression. These results provided strong evidence that AtAN is the transcriptional regulator of PEP shunt between shikimate and pyruvate biosynthesis.

### Example 3

### AtAN Represses the Shikimate Pathway and Preferentially Shuttles Carbon Towards Pyruvate Biosynthesis

Differential expression of transcription factors associated with JA and ET signaling were examined. As predicted, five WRKY transcription factors and seven ethylene response factors (ERFs) previously implicated in JA/ET signaling exhibited >2-fold decrease in expression in the mutant line (FIG. 1).

Additionally, the signal transducer NPR1-like protein was similarly down-regulated. Since AtAN is proposed to function as a transcriptional repressor, these results suggest that in its functional state, AtAN represses the Shikimate pathway and preferentially shuttles carbon towards pyruvate biosynthesis.

### Example 4

### The Nuclear Localization of AN

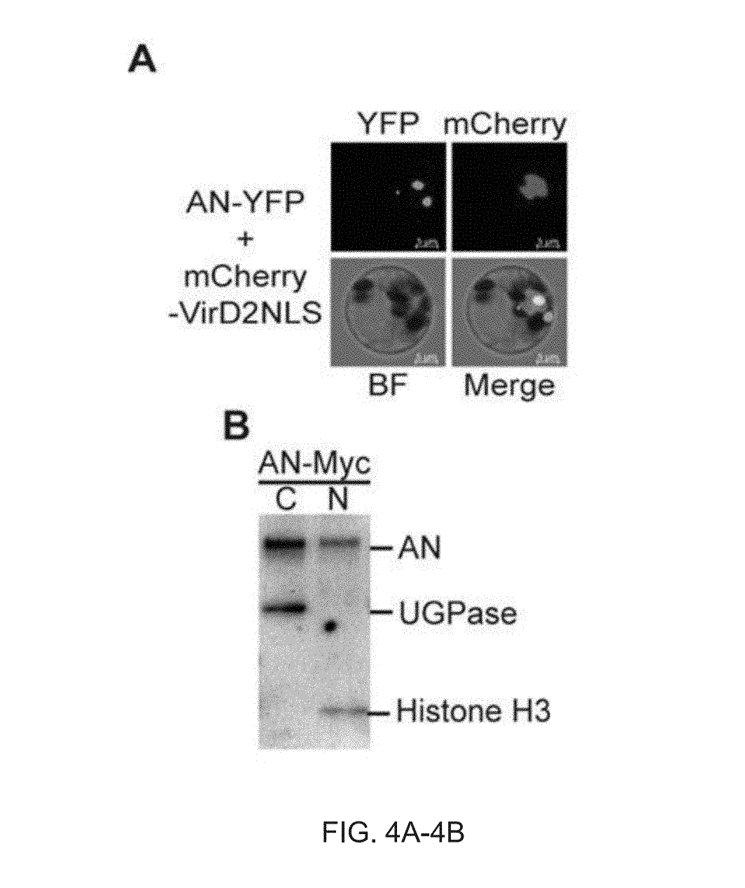

To determine whether AN has nuclear functions, the inventors examined the subcellular localization of Yellow Fluorescent Protein (YFP)-tagged AN (AN-YFP) using the Arabidopsis mesophyll protoplast transient expression system. As shown in FIG. 4A, signals of AN-YFP (green color) are present in both the cytoplasm and the nucleus. In 38.75% transfected cells (N=80), signals of AN-YFP partially overlap with signals of the nuclear marker mCherry-VirD2NLS (Lee et al., Plant Methods, 4, 24 (2008)), as indicated by the yellow color (FIG. 4A). Consistent with previously published work (Minamisawa et al., Plant J., 68, 788-799 (2011)), signals of AN-YFP in the cytoplasm partially overlap with Golgi-mCherry marker. In addition, in the protoplasts, Myc tagged AN (AN-Myc) is detected in both cytosolic and nuclear fractions using Western blotting (FIG. 4B), further supporting the nuclear accumulation of AN protein.

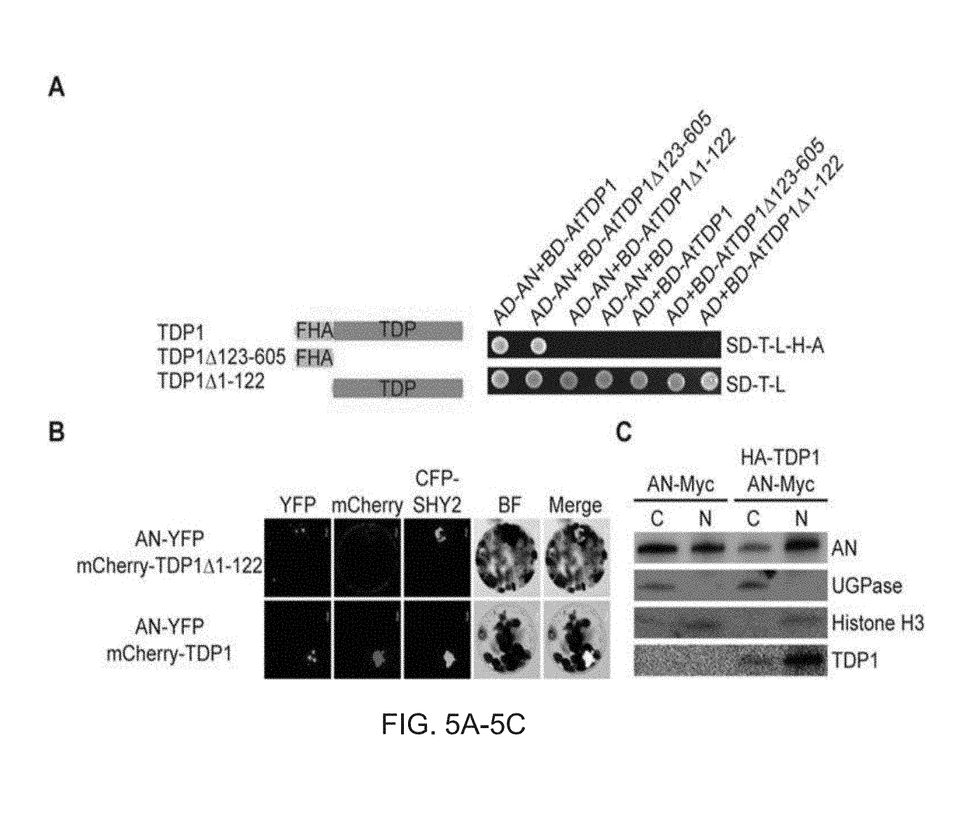

The nuclear localization signal (NLS; KKRH) in the C-terminal region of AN was believed to be unrelated to AN function because Arabidopsis an mutant phenotypes can be complemented by Marchantia polymorpha ANGUSTIFOLIA, which lacks an NLS (Minamisawa et al., Plant J., 68, 788-799 (2011)). To determine the mechanism guiding AN into the nucleus, the inventors sought to identify proteins interacting with AN. Gachomo et al. identified nine potential interacting partners of AN using the high-throughput integrated knowledge-based Arabidopsis protein interaction network analysis (ANAP) (Gachomo et al., Bmc Plant Biology, 13, 1 (2013)). Among these nine proteins, only At5G15170 (TDP1) has been reported to localize in the nucleus (Lee et al., Plant Physiol., 154, 1460-1469 (2010)), which prompted the inventors to determine its interaction with AN. As shown in FIG. 5A, the interaction between AN and TDP1 was confirmed using the yeast two-hybrid (Y2H) assay. Consistent with the Y2H result, AN-YFP showed co-localization with mCherry-TDP1 in both the cytoplasm and the nucleus (FIG. 5B). TDP1 is a key enzyme catalyzing DNA repair in both plants and animals (Ledesma et al., Nature, 461, 674-U125 (2009); Lee et al., Plant Physiol., 154, 1460-1469 (2010)). In addition to the TDP domain that is present in all TDP1 proteins, Arabidopsis TDP1 contains an FHA domain at its N-terminal region (Kim et al., EMBO J 21, 1267-1279 (2012)). The FHA domain of TDP1 contains an NLS and is indispensable for its nuclear localization (Kim et al., EMBO J 21, 1267-1279 (2012). The inventors' Y2H and co-localization analyses of AN and truncated TDP1 demonstrated that AN specifically interacts with the FHA domain (TDP1Δ123-605), whereas has no interaction with the TDP domain (TDP1Δ1-122) (FIGS. 5A and 5B).

Given the result herein that AN interacts with the NLS-containing domain of TDP1 (FHA domain), TDP1 may be involved in the nuclear accumulation of AN. To test this hypothesis, AN-Myc and HA-tagged TDP1 (HA-TDP1) were co-expressed in the protoplasts and their accumulations in the cytoplasm and nucleus were measured using Western blotting. Reduced cytosolic accumulation and increased nuclear accumulation of AN were observed when AN-Myc and HA-TDP1 were co-transfected (FIG. 5C), suggesting that TDP1 enhances the nuclear localization of AN. Moreover, the presence of a small portion of TDP1 protein in the cytosolic fraction (FIG. 5C) implies that TDP1 may bind to AN in the cytoplasm and guide AN into the nucleus.

### Example 5

### AN has Transcriptional Repressor Activity and Targets the Transcription Factor Gene MYB46

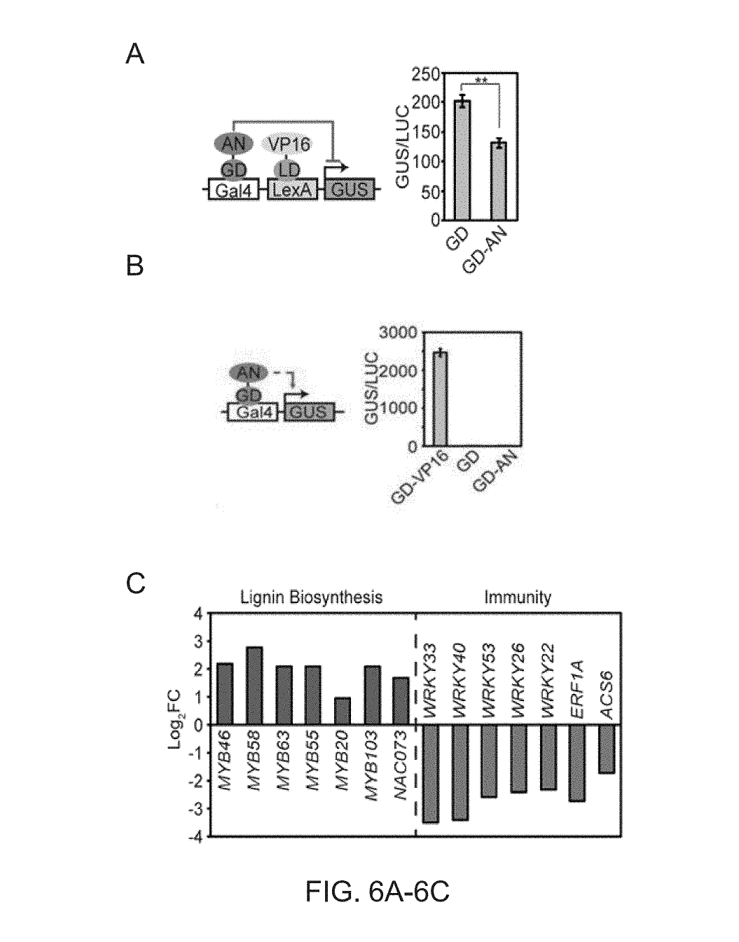

The nuclear accumulation of AN suggests potential transcriptional functions. The inventors then analyzed whether AN can regulate transcription by using the transactivation assays. In this assay, the promoter region of ’-glucuronidase (GUS) reporter gene contains two DNA binding sites, Gal4 and LexA (FIG. 6A). LexA-binding-domain (LD)-fused Herpes simplex virus VP16 (LD-VP16) (Tiwari et al., Plant Cell, 16, 533-543 (2004)) was used to constitutively activate GUS expression (FIG. 6A). The results that Gal4-binding-domain (GD)-fused AN (GD-AN), but not GD only, reduced GUS expression suggested that AN has transcriptional repressor activity (FIG. 6A). No transcriptional activator activity of AN was detected (FIG. 6B).

Subsequently, target genes of AN were identified using micro chromatin immunoprecipitation (μChIP) (Dahl and Collas, Nature Protocols, 3, 1032-1045 (2008)) following quantitative PCR (qPCR). Given that the expression of a number of transcription factor genes were altered in an-1 knockout mutant (Bryan et al., Genes & Development, 18, 1577-1591(2018)), the inventors hypothesized that AN may regulate the transcription of transcription factor genes. The inventors focused on transcription factors with significant gene expression changes (>2 folds) in an-1 (FIG. 6C). Among 12 tested transcription factors (Seven up-regulated genes: MYB58, MYB46, MYB63, MYB55, MYB20, MYB103, and NAC073; Five down-regulated genes: WRKY33, WRKY40, WRKY53, WRKY26, and WRKY22), AN only showed association with the MYB46 promoter in the ChIP assays (FIG. 6D and FIG. 6E). An in vitro DNA binding assay (electrophoretic mobility shift assay, EMSA) was then used to determine the direct binding of AN to the MYB46 promoter. Purified GST-tagged AN protein (GST-AN), but not the GST tag alone, was found to bind to the biotin-labeled 148-bp MYB46 promoter fragment (−447-bp to −300-bp) (FIG. 6F), suggesting that AN has DNA binding activity and directly targets MYB46. A competition assay using 100×unlabeled MYB46 promoter DNA abolished the shifted band, suggesting that the binding of AN to MYB46 promoter is specific (FIG. 6F).

### Example 6

### TDP1 Enhances the Transcriptional Function of AN

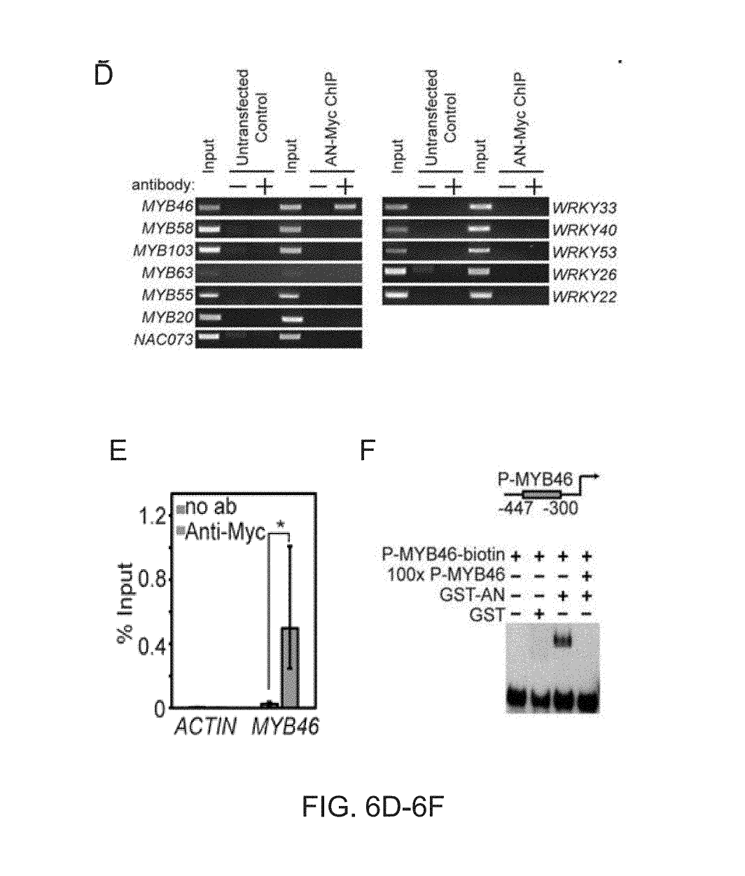

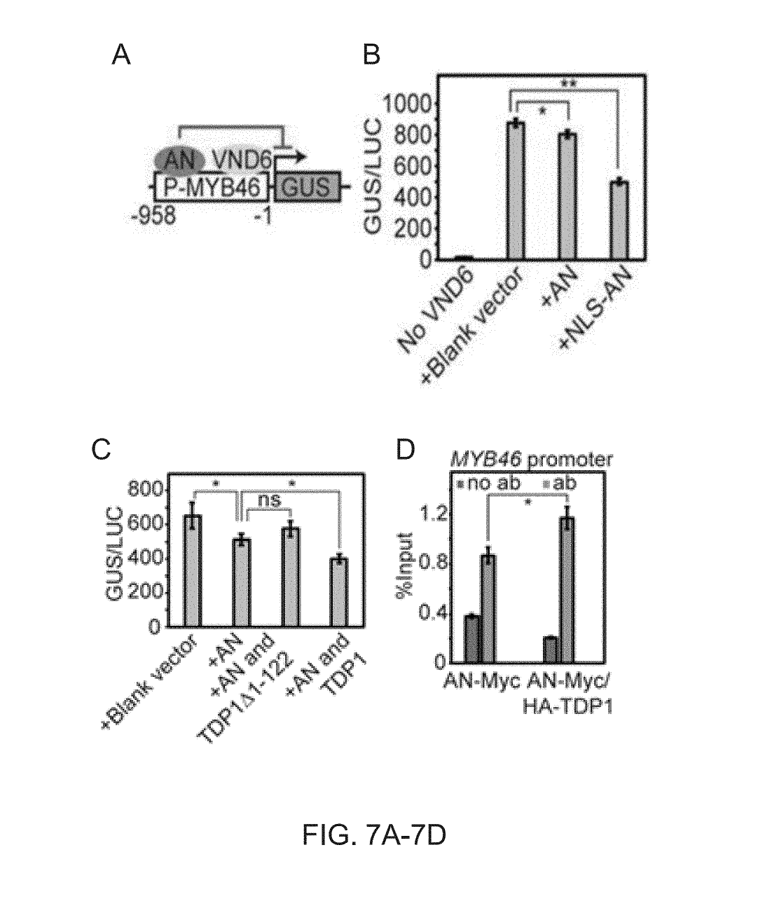

To determine the effect of AN on MYB46 expression, a transactivation assay was performed. Because AN was shown to act as a transcriptional repressor (FIG. 6A), VND6, a transcriptional activator for the MYB46 gene (Zhong et al., Plant Cell, 19, 2776-2792 (2007)) was used to constitutively activate MYB46 expression in the protoplast transient expression system. As shown in FIG. 7B, NLS-fused AN (NLS-AN) exhibited stronger repression on MYB46 promoter activity (indicated as a reduction in the GUS reporter activity) than AN, suggesting that the nuclear localization of AN is critical for repression of MYB46 (FIG. 7B). Consistent with this observation, full-length TDP1, not the truncated TDP1 lacking the FHA domain (TDP1Δ1-122), enhanced the repression of AN on MYB46 in the transactivation assay (FIG. 7C).

Because TDP1 enhanced the nuclear accumulation of AN and enhanced its repressor activity on MYB46, we wanted to investigate whether TDP1 could increase the chromatin association of AN. AN-Myc and HA-TDP1 were co-expressed in the protoplasts and subjected to μChIP-qPCR assay. The protein level of AN-Myc remained unchanged with and without TDP1 co-expression, suggesting that TDP1 does not affect the stability or turnover of AN protein. The association between AN and MYB46 promoter was measured with qPCR amplifying MYB46 promoter DNA from immunoprecipitated products. Consistent with the increased nuclear accumulation, the chromatin association of AN with MYB46 promoter was significantly increased (p<0.05) in vivo when co-expressed with TDP1 (FIG. 7D).

Collectively, these results indicate that AN has a transcriptional repressor function and targets MYB46 and that the transcriptional repressor function requires the interaction with TDP1.

### Example 7

### The AN-TDP1 Interaction Releases the Transcriptional Repression of TDP1 on the Transcription Factor Gene WRKY33

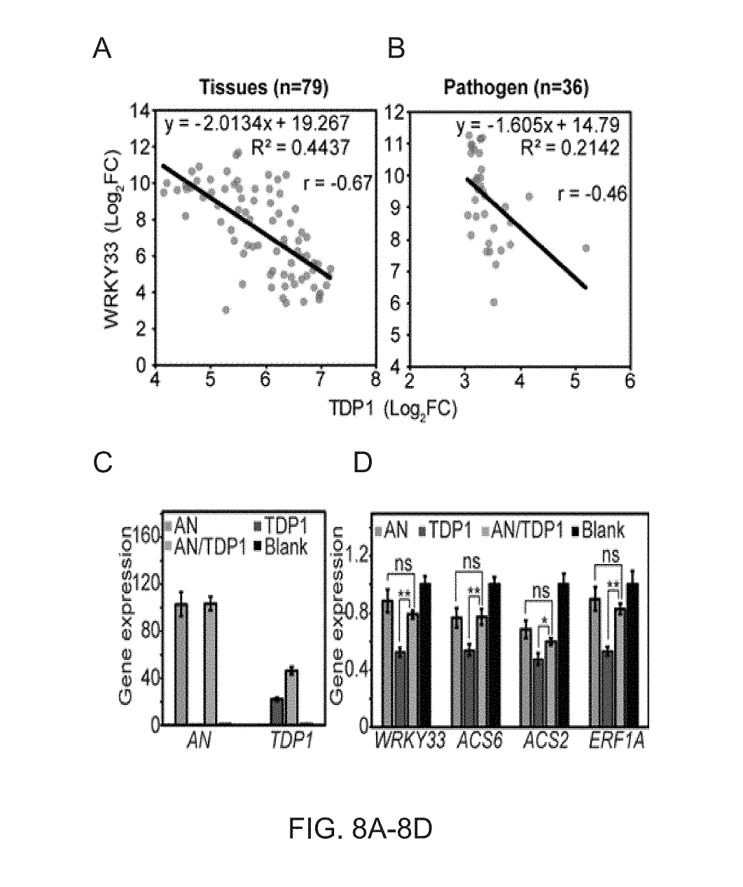

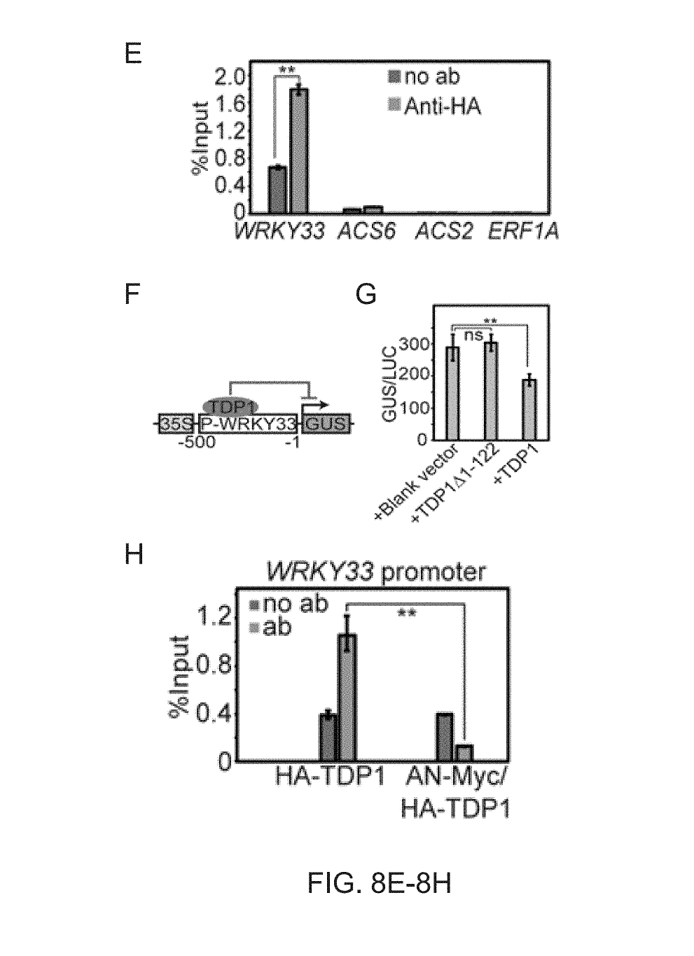

Among plant TDP1s, only the transcriptional effect of Medicago TDP1 has been analyzed (Dona et al. J Exp Bot, 64, 1941-1951 (2013)). Antagonistic to gene expression changes observed in the Arabidopsis an-1 mutant, deletion of Medicago TDP1 activated genes involved in immunity and ethylene pathways, including WRKY DNA-BINDING PROTEIN 33 (WRKY 33) and ETHYLENE RESPONSIVE ELEMENT BINDING FACTOR 1 (ERF1A) (Dona et al., J Exp Bot, 64, 1941-1951, (2013)). Given Arabidopsis TDP1 (AT5G15170) and Medicago TDP1 (XM_003622639) proteins share high amino acid identity (62.8%) and a similar structure (both of them have the FHA domain and TDP domain), the inventors hypothesized that Arabidopsis TDP1 may negatively regulate the expression of immunity genes, such as WRKY33 and ERF1A. To test this hypothesis, the inventors analyzed the expression patterns of Arabidopsis TDP1 and WRKY33 and found that they displayed negative co-expression across different developmental stages and pathogen infections (FIG. 8B), supporting the hypothesis of a close relationship between TDP1 and WRKY33. The inventors further tested this using the protoplast transient expression system. Overexpression of Arabidopsis TDP1 was found to reduce transcript levels of endogenous WRKY33, ERF1A, as well as ACC SYNTHASE 2 and 6 (ACS2 and ACS6), two genes directly activated by WRKY33 (Datta et al., Plant Physiol., 169(4):2963-81, (2015)) (FIG. 8D). The inventors then performed μChIP-qPCR to determine whether Arabidopsis TDP1 associates with promoters of these four genes (WRKY33, ACS2, ACS6, and ERF1A) to modulate their transcription. As shown in FIG. 8E, among these four tested genes, TDP1 only exhibited significant association with the WRKY33 promoter. Because ACS2/6 catalyze the rate-limiting step of ethylene biosynthesis (Liu and Zhang, Plant Cell, 16, 3386-3399 (2004)), which subsequently induces ERF1 expression, it is possible that TDP1 negatively modulates WRK133 expression to affect its downstream pathways. To further test the transcriptional repression of Arabidopsis TDP1 on WRKY33, TDP1 was co-transfected with GUS reporter which was placed downstream of the Cauliflower mosaic virus 35S (CaMV 35S) promoter and the WRKY33 promoter (FIG. 8F). The reduced GUS reporter activity observed in this assay confirmed the hypothesis that the promoter activity of WRKY33 is suppressed by TDP1, but not by FHA-deleted TDP1 (FIG. 8G).

As shown above (FIG. 7C), the AN-TDP1 interaction enhanced AN's transcriptional repression of MYB46. The inventors sought to investigate whether the AN-TDP1 interaction also affects the transcriptional function of TDP1. To test this, transcript levels of WRKY33, ACS2, ACS6, and ERF1 were compared in the protoplasts overexpressing TDP1 only with those co-overexpressing AN and TDP1. As shown in FIGS. 8C and 8D, co-expression of AN and TDP1 released TDP1's transcriptional repressions of these four genes and restored gene expression to levels of expressing AN only (FIGS. 8C and 8D), suggesting AN-TDP1 interaction negatively affects TDP1-induced transcriptional repression.

To further investigate the underlying mechanism, the inventors assessed the chromatin association changes of TDP1 in the presence of AN (FIG. 8H). It was found that the association of TDP1 with the WRKY33 promoter was significantly reduced (p<0.01) when co-expressed with AN, suggesting that AN can positively regulate WRKY33 expression via releasing its transcriptional repression imposed by TDP1.

### Example 8

### AN Modulates Plant Resistance Against Botrytis cinerea Via Transcriptional Reprogramming

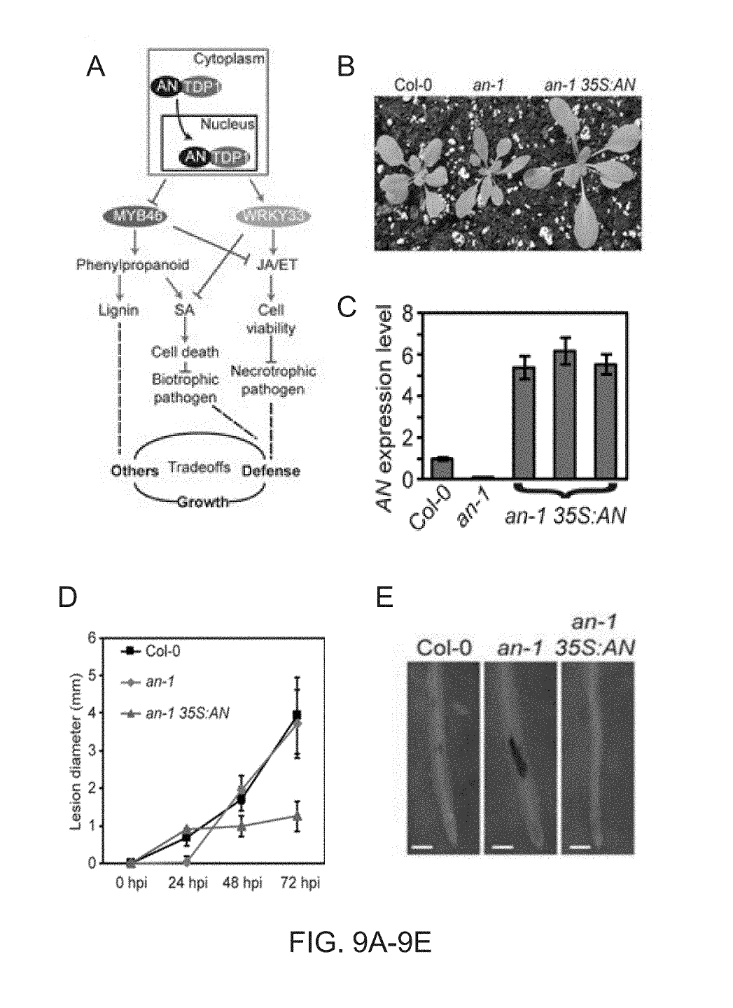

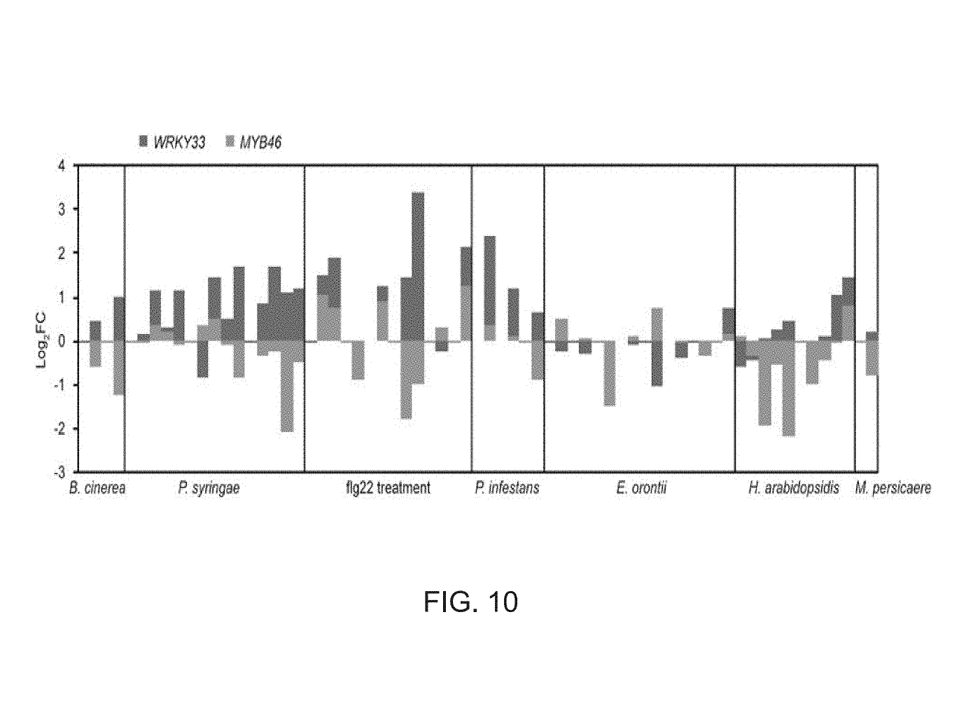

On the basis of the molecular and biochemical characterizations described above, we proposed a model of action of AN in the transcriptional regulation (FIG. 9A). In conjunction with TDP1, AN protein accumulates in the nucleus and represses MYB46 expression. Moreover, by interacting with TDP1, AN releases the transcriptional repression of WRKY33. Consequently, AN antagonistically regulates the expression of MYB46 and WRKY33 (FIG. 9A).

The transcription factors MYB46 and WRKY33 have been reported to associate with plant defense against fungal pathogen Botrytis cinerea (B. cinerea) and trigger inducible immunity responses (Zheng et al, Plant Journal, 48, 592-605 (2006); Ramirez et al., Plant Physiol, 155, 1920-1935 (2011)). However, these two transcription factors seem to act oppositely during B. cinerea infection. As a result, wrky33 knockout mutants are susceptible to B. cinerea (Zheng et al., Plant Journal, 48, 592-605 (2006)), whereas myb46 mutants are resistant to B. cinerea (Ramirez et al., Plant Physiol, 155, 1920-1935 (2011)). Because AN antagonistically regulates the expression of MYB46 and WRKY33, the inventors sought to determine whether AN affects plant response to B. cinerea.

B. cinerea inoculation was performed using leaves from an-1 and AN overexpression plants (an-1 35S:AN; AN expression is five to six folds of that of Col-0; FIG. 9C). Based on the inventor's model of action of AN, AN overexpression inhibits MYB46 expression and enhances WRKY33 expression, which would promote the resistance against B. cinerea. On the other hand, loss of AN would have the opposite transcriptional effect and result in increased susceptibility to B. cinerea. As expected, during the late stage (48 to 72 hpi), an-1 35S:AN plants displayed resistance to B. cinerea whereas an-1 plants exhibited rapid progress of B. cinerea infection, which was indicated by the quickest lesion diameter increase from 48 to 72 hpi (FIG. 9D). In addition to the resistance to B. cinerea, an-1 35S:AN plants had larger rosette size with larger leaves than wild type plants whereas the opposite growth phenotype was observed in the an-1 mutants (FIG. 9B). To obtain a global view of the relationship between MYB46 and WRKY33 under biotic stresses, the inventors compared the transcript levels of these two genes based on existing microarray and RNA-seq datasets in the Arabidopsis eFP Browser (bar.utoronto.ca) (Winter et al., PLoS One, 2, e718 (2007)), MYB46 and WRKY33 exhibited opposite expression patterns under various biotic stresses, including infections with B. cinerea, Pseudomonas syringae, Phytophthora infestans, Erysiphe orontii, Hyaloperonospora arabidopsidis, herbivore stress (Myzus persicaere) and flg22 treatment. These results suggest that, in addition to responses to B. cinerea infection, the mechanism of AN-regulated transcriptional reprogramming of MYB46 and WRKY33 may be functional during other biotic stresses.

During early stage of infection (0 to 24 hpi), we observed one interesting phenomenon that an-1 35S:AN plants were susceptible to B. cinerea whereas an-1 plants were resistant to B. cinerea, which is a completely opposite pattern from that during the late stage (48 to 72 hpi). As noted above, MYB46 is a master activator of lignin biosynthesis, which is a critical component of the first barrier against pathogen infection. The model of action of AN proposed herein indicates that MYB46 is up-regulated in an-1 and down-regulated in an-1 35S:AN. Consequently, an-1 would have more lignin deposition than an-1 35S:AN which may explain how an-1 is more resistant to B. cinerea than an-1 35S:AN during early stage of infection. Consistent with this view, ectopic patches of lignin were observed in an-1 mutants, which were absent in an-1 35S:AN (FIG. 9E).

### Example 9

### Discussion

In animals, CtBP not only functions as a critical transcriptional co-repressor promoting tumorigenesis (Chinnadurai, Cancer Res, 69, 731-734 (2009)), but also controls the partition of Golgi during mitosis in the cytoplasm (Carcedo et al., Science, 305, 93-96 (2004)). However, it has long been thought that ANGUSTIFOLIA only has cytosolic function and is involved in microtube-related processes (Minamisawa et al., Plant J., 68, 788-799 (2011); Kwak et al., Biochem Biophys Res Commun., 465, 587-593 (2015)). In a previous report, localization analyses of AN using stable transgenic plants expressing green florescent protein-tagged AN failed to detect AN in the nucleus (Minamisawa et al., Plant J., 68, 788-799 (2011)). However, the possibility of AN nuclear location could not be ruled out because AN may have rapid nucleocytoplasmic shuttling as CtBP2 (Zhao et al., J Biol Chem., 281, 4183-4189 (2006)), which forms occasional nuclear localization and may result in the lack of nuclear localization in stable transgenic plants. The localization and cell fraction analyses using the transient expression system demonstrated herein the nuclear accumulation of AN. More importantly, the present inventors identified one AN interacting partner, which has the capability to enhance AN nuclear accumulation. Although the NLS in AN protein seems nonfunctional (Minamisawa et al., Plant J, 68, 788-799 (2011)), AN physically associates with TDP1's FHA domain, which contains a functional NLS (Kim et al., Biochem J, 443, 49-56 (2012)). In addition, the cell fraction analysis (FIG. 4) and subcellular localization result in an earlier report (Lee et al., Plant Physiol., 154, 1460-1469 (2010)) demonstrate that TDP1 does not solely localize in the nucleus and that a small fraction of TDP1 protein is present in the cytosol. Together, these results provide evidence that AN has nuclear accumulation and may have nuclear functions similar to CtBP. Consequently, the inventors observed transcriptional repressor activity of AN in the transactivation assays and identified transcription factor MYB46 gene as the direct target of AN. Drosophila and mammalian CtBPs do not bind to DNA directly, but depend on associating with sequence-specific DNA-binding transcription factors to determine its targets (Byun and Gardner, Int J Cell Biol., 2013:647975 (2013)). The results disclosed in this application suggest that AN may have a different target recognition mechanism due to its DNA binding capability. Therefore, the inability of using AN to complement a CtBP loss-of-function mutant in Drosophilia may be due to target differences.

During tumorigenesis, CtBP maintains the viability of cancer cells by suppressing the expression of apoptosis signaling (Chinnadurai, Cancer Res., 69, 731-734 (2009)). In this study, the inventors find that AN can enhance plant cell viability during B. cinerea infection by releasing transcriptional repression of transcriptional factor gene WRKY33 which in turn activates JA-induced defense (Birkenbihl et al., Plant Physiology, 159, 266-285 (2012)) to fight against necrotrophic pathogens (Spoel et al., PNAS, 104, 18842-18847 (2007)). B. cinerea has been found to trigger plant hypersensitive response (HR), which generates reactive oxygen intermediates (ROIs) such as H₂O₂ to activate hypersensitive cell death (Govrin and Levine, Curr Biol., 10, 751-757 (2000)). Given the previously published result that an knockout mutants exhibited two times higher H₂O₂ accumulation than wild type plants (Gachomo et al., Bmc Plant Biology, 13, 1 (2013)), it is plausible that AN can inhibit plant cell death during necrotrophic pathogen infection by preventing the accumulation of H₂O₂. Although the downstream molecular mechanisms are different, CtBP and AN still maintain a similar role in cell death control. Such analogous roles of CtBP and AN demonstrate that the biological role of one protein may still be conserved even after millions of years of evolution, which highlights the importance of comparative studies of plant and animal genes.

A key finding of this study is the identification of a transcriptional machinery antagonistically modulating lignin biosynthesis and defense against B. cinerea. Current frameworks of growth-defense mechanisms merely consider plant metabolism as either growth or defense. However, the variety of plant metabolisms argues for the existence of a grey area, where metabolisms cannot be easily classified into growth or defense. One example is the biosynthesis of lignin, which not only provides structural support for growth but also protects plant cells against pathogens. On the other hand, the fact that lignin is energy rich and the biosynthesis of lignin is resource-consuming provides a potential toggle mechanism to mitigate the tradeoff between growth and defense. However, current studies on the transcriptional network controlling growth-defense tradeoffs do not include such a scenario. The inventors' discovery of antagonistic regulation of lignin biosynthesis and B. cinerea defense by AN fills the gap. The antagonistic transcriptional regulation of MYB46 and WRKY33 demonstrates the existence of a transcriptional center for the tradeoff between lignin biosynthesis and B. cinerea defense. Although WRKY transcription factors were thought to be involved in plant defense, WRKY12 has been demonstrated to repress lignin biosynthesis in pith cells (Wang et al., PNAS, 107, 22338-22343 (2010)). Whether WRKY33 is capable of directly affecting lignin biosynthesis remains an open question. On the other hand, lignin biosynthesis and growth also have a tradeoff because overexpression of multiple master regulators of lignin biosynthesis in Arabidopsis was found to inhibit growth (Zhong et al., Plant Cell, 19, 2776-2792 (2007); Zhou et al., Plant Cell, 21, 248-266 (2009)). This is consistent with the observation in the present study in which an-1 mutant was found to have ectopic lignin deposition together with reduced plant growth.

The present findings suggest that the role of MYB46 in the biosynthesis of cell wall is also important to fight against B. cinerea infection, especially during the early stage of infection. an-1 plants displayed increased MYB46 expression and ectopic lignin deposition, which co-occur with B. cinerea resistance during the early stage of infection (0 to 24 hpi). Because up-regulation of MYB46 has been reported to enhance lignin deposition in leaf cells (Zhong et al., Plant Cell, 19, 2776-2792 (2007)), it is possible that MYB46 is capable of strengthening leaf cell walls to block pathogen infection by providing a chemical or physical barrier. The antagonistic roles of MYB46 in the formation of a chemical or physical barrier and activation of induced immunity responses may explain how overexpression of MY1346 did not exhibit any altered disease susceptibility to B. cinerea in a previous study (Ramirez et al., Plant Physiol, 155, 1920-1935 (2011)). This illustrates the complex relationship between cell wall lignification and plant defense capability, as observed in an earlier study (Zhao and Dixon, Annu Rev Phytopathol, 52, 69-91 (2014)).

Biotrophic pathogens need to keep plant cells alive as a long-term nutritional resource. In contrast, neurotrophic pathogens kill plant cells to obtain nutrition (Glazebrook, Annu Rev Phytopathol., 43, 205-227 (2005)). WRKY33 was found to regulate defense against necrotrophic and biotrophic pathogens oppositely by activating JA (activating defense against necrotrophic pathogens) but inhibiting salicylic acid signaling (activating defense against biotrophic pathogens). Consistently, the an mutant was found to be resistant to the infection of Pseudomonas syringae (Gachomo et al., Bmc Plant Biology, 13, 1 (2013)), which is a biotrophic pathogen.

Collectively, the present disclosure reveals a transcriptional nexus to modulate resource partitioning between lignin biosynthesis and B. cinerea defense. This finding may inform biosystems design of effective and efficient strategies to mitigate the tradeoffs between growth and defense and enhance crop performance and fitness under stress conditions.

What is claimed is:

1. A method comprising modulating the ANGUSTIFOLIA gene expression in a plant.

2. The method of claim 1, wherein the modulation of the ANGUSTIFOLIA gene expression comprises inactivation of the ANGUSTIFOLIA gene in the plant.

3. The method of claim 2, wherein the inactivation of the ANGUSTIFOLIA gene is achieved by introducing a nucleic acid inhibitor of the ANGUSTIFOLIA gene to the plant.

4. The method of claim 3, wherein the nucleic acid inhibitor is selected from the group consisting of an antisense RNA, a small interfering RNA, an RNAi, a microRNA, an artificial microRNA, and a ribozyme.

5. The method of claim 2, wherein the inactivation of the ANGUSTIFOLIA gene is achieved by genome editing, which is achieved by a method selected from the group consisting of CRISPR/Cas system, Cre/Lox system, TALEN system, ZFNs system and homologous recombination.

6. The method of claim 5, wherein the CRISPR-mediated genome editing comprises introducing into the plant a first nucleic acid encoding a Cas9 nuclease, a second nucleic acid comprising a guide RNA (gRNA), wherein said gRNA is specific to the ANGUSTIFOLIA gene.

7. The method of claim 1, wherein said plant is a member of the genus selected from the group consisting of Acer, Afzelia, Arabidopsis, Betula, Brassica, Eucalyptus, Fagus, Fraxinus, Glycine, Gossypium, Jatropha, Juglans, Linum, Lycopersicon, Medicago, Micropus, Populus, Prunus, Quercus, Salix, Solanum, Tectona, Trifolium, Agrostis, Avena, Festuca, Hordeum, Lemna, Lolium, Milium, Miscanthus oryza, Panicum, Pennisetum, Phalaris, Phleum, Poa, Saccharum, Secale, Sorghum, Triticum, Zea, Zoysia, Abies, Picea and Pinus.

8. The method of claim 7, wherein said plant is selected from the group consisting of Festuca arundinacea, Miscanthus giganteus, Miscanthus sinensis, Miscanthus sacchariflorus, Panicum virgatum, Pennisetum purpureum, Phalaris arundinacea, Populus balsamifera, Populus deltoides, Populus tremuloides, Populus tremula, Populus alba, Populus maximowiczii, Saccharum officinarum, Saccharum ravennae, Secale cereale, Sorghum almum, Sorghum halcapense, and Sorghum vulgare.

9. The method of claim 1, wherein the modulation of the ANGUSTIFOLIA gene expression comprises expressing an exogenous nucleic acid encoding an ANGUSTIFOLIA gene in the plant.

10. The method of claim 9, wherein the exogenous nucleic acid is stably transfected or transformed into the plant genome.

11. A plant, wherein the expression of the ANGUSTIFOLIA gene is modulated and altered as compared to a plant in which the ANGUSTIFOLIA gene is not modulated.

12. The plant of claim 11, wherein the modulation comprises inactivation of the ANGUSTIFOLIA gene in the plant.

13. The plant of claim 11, wherein the modulation comprises expressing an exogenous nucleic acid encoding an ANGUSTIFOLIA gene in the plant.

14. The plant of claim 11, wherein the plant is a member of the genus selected from the group consisting of Acer, Afzelia, Arabidopsis, Betula, Brassica, Eucalyptus, Fagus, Fraxinus, Glycine, Gossypium, Jatropha, Juglans, Linum, Lycopersicon, Medicago, Micropus, Populus, Prunus, Quercus, Salix, Solanum, Tectona, Trifolium, Agrostis, Avena, Festuca, Hordeum, Lemna, Lolium, Milium, Miscanthus oryza, Panicum, Pennisetum, Phalaris, Phleum, Poa, Saccharum, Secale, Sorghum, Triticum, Zea, Zoysia, Abies, Picea and Pinus.

15. The plant of claim 14, wherein the plant is selected from the group consisting of Festuca arundinacea, Miscanthus gigantetts, Miscanthus sinensis, Miscanthus saccharylorus, Panicum virgatum, Pennisetum purpureum, Phalaris arundinacea, Populus balsamifera, Populus deltoldes, Populus tremuloides, Populus tremula, Populus alba, Populus maximowiczii, Saccharum officinarum, Saccharum ravennae, Secale cereale, Sorghum almum, Sorghum halcapense, and Sorghum vulgare.

16. A method for producing a bioproduct, comprising subjecting the plant of claim 11 to a bioproduct conversion process.

17. The method of claim 16, wherein the bioproduct is selected from the group consisting of a bioenergy product, a biomaterial, a biopharmaceutical and a biocosmetics.

18. The method of claim 17, wherein the bioenergy product is ethanol and the bioproduct conversion process is an ethanol fermentation process.

19. The method of claim 17, wherein the bioproduct is selected from the group consisting of ethanol, biodiesel, biogas, bioplastics, biofoams, biorubber, biocomposites, and biofibres.

20. A method for production of pulp or paper, comprising producing pulp or paper from the plant of claim 11.


SEQUENCE LISTING (56 sequences)

SEQ ID NO: 1 - protein, 636 aa, Arabidopsis thaliana
MSKIRSSATMPHRDQPSPASPHVVTLNCIEDCALEQDSLAGVAGVEYVPLSRIADGKIESATAVLLHSLAYLPRAAQRRLRPHQLILCLGSADRAVDSTLAADLGLRLVHVDTSRAEEIADTVMALILGLLRRTHLLSRHALSASGWLGSLQPLCRGMRRCRGMVLGIVGRSVSARYLASRSLAFKMSVLYFDVPEGDEERIRPSRFPRAARRMDTLNDLLAASDVISLHCALTNDTVQILNAECLQHIKPGAFLVNTGSCQLLDDCAVKQLLIDGTIAGCALDGAEGPQWMEAWVKEMPNVLILPRSADYSEEVWMEIREKAISILHSFFLDGVIPSNTVSDEEVEESEASEEEEQSPSKHEKLAIVESTSRQQGESTLTSTEIVRREASELKESLSPGQQHVSQNTAVKPEGRRSRSGKKAKKRHSQQKYMQKTDGSSGLNEESTSRRDDIAMSDTEEVLSSSSRCASPEDSRSRKTPLEVMQESSPNQLVMSSKKFIGKSSELLKDGYVVALYAKDLSGLHVSRQRTKNGGWFLDTLSNVSKRDPAAQFIIAYRNKDTVGLRSFAAGGKLLQINRRMEFVFASHSFDVWESWSLEGSLDECRLVNCRNSSAVLDVRVEILAMVGDDGITRWID

SEQ ID NO: 2 - DNA, 1911 bp, Arabidopsis thaliana
atgagcaagatccgttcgtctgcgacaatgccacatcgcgaccagccgtcaccggcgtcgcctcacgtcgttacactcaactgtatagaggattgtgcgctcgagcaagactccctcgccggcgttgctggtgtcgaatacgtcccgctcagccgcatcgccgatggtaagatcgagtccgccaccgccgttctcctccattccctcgcgtatcttccacgagcagctcaacgccgactccgtcctcaccagctcattctctgccttggctctgctgatcgcgctgtcgattcgactctcgccgccgacctaggtctccgacttgtccatgtagatacttcacgagccgaggaaatcgcggataccgtcatggcgctcatccttggactccttcgacggacgcatttactctcgcgacacgctctatcggcgtctggttggctcggatcgcttcagcctctttgccggggaatgagacggtgccgtggtatggttttgggtatcgttggcagatctgtatcggctcggtatttagctagtagaagcttggctttcaagatgagtgtgctctacttcgatgtcccagagggagatgaagaacgaatcaggccctcgagattcccacgtgctgctcgaagaatggatacattgaatgatcttctagcagcaagtgatgtcatttcgctacattgtgcattaacaaatgacacggttcagatactcaatgcagagtgtttgcagcatataaaacctggggcttttcttgtaaatactggaagctgccagctgttggatgattgtgctgtgaaacaacttctaattgatggcactatagctggctgcgcccttgacggtgctgaaggtccacaatggatggaagcatgggtgaaggaaatgccaaatgtgttaattctacctcgcagtgcagattacagtgaggaagtatggatggagataagggagaaggctatctctatcttgcattcatttttcttagatggtgtaattccaagtaacactgtttctgatgaggaagttgaggaaagtgaagcaagtgaagaagaagaacaatcacctagcaaacacgagaaattagcaatagtggaatccaccagtaggcaacagggagaaagtactctcaccagcactgagatcgtacgtagagaggctagtgagttaaaagaatctctgagccctggtcagcaacacgtttctcaaaatactgccgtaaaacctgaaggaagacgtagcagatccggtaagaaagccaaaaagagacattcacagcaaaaatacatgcaaaaaacggatggttcctcagggttaaatgaagaaagtacttcacgaagagatgatattgctatgagtgacacagaagaagtattaagttccagttctagatgtgcttctcctgaagattccagaagtaggaaaacacctcttgaagtaatgcaagagtcttccccaaatcagcttgtaatgtcaagtaagaagttcattggaaagtcaagtgagctactgaaagatggatatgtagtagccttgtatgcgaaagacctctcgggcctccacgtttccaggcaaagaacgaaaaacggtggctggttcctcgatactttgtccaatgtatccaaacgagatcctgctgcacaattcattatcgcatacagaaacaaggacactgttggtctgagatcatttgctgctggtgggaagttactgcagatcaatagaagaatggagtttgtgtttgctagccatagttttgacgtgtgggagagttggagtctagaaggttctctggacgaatgtcggcttgttaactgcaggaattcctctgcggtgttggacgttcgtgtggagatattggcaatggtaggagacgatggtatcacacgttggatcgattaa

SEQ ID NO: 3 - DNA, 26 bp, Artificial Sequence
  Oligonucleotide
caccatgagcaagatccgttcgtctg

SEQ ID NO: 4 - DNA, 22 bp, Artificial Sequence
  Oligonucleotide
ttaatcgatccaacgtgtgata

SEQ ID NO: 5 - DNA, 26 bp, Artificial Sequence
  Oligonucleotide
caccatggctcactctcaggttgctt

SEQ ID NO: 6 - DNA, 30 bp, Artificial Sequence
  Oligonucleotide
atgtcgacctatcttggccagacttgtcca

SEQ ID NO: 7 - DNA, 26 bp, Artificial Sequence
  Oligonucleotide
caccaaaaggagtataacattttctt

SEQ ID NO: 8 - DNA, 22 bp, Artificial Sequence
  Oligonucleotide
ctatcttggccagacttgtcca

SEQ ID NO: 9 - DNA, 26 bp, Artificial Sequence
  Oligonucleotide
cacccaaactcacttctaaatactaa

SEQ ID NO: 10 - DNA, 22 bp, Artificial Sequence
  Oligonucleotide
ctgtatatttgttggttatgtc

SEQ ID NO: 11 - DNA, 20 bp, Artificial Sequence
  Oligonucleotide
gtcttccccaaatcagcttg

SEQ ID NO: 12 - DNA, 20 bp, Artificial Sequence
  Oligonucleotide
ccgagaggtctttcgcatac

SEQ ID NO: 13 - DNA, 20 bp, Artificial Sequence
  Oligonucleotide
ttacctggccgtggtaatgt

SEQ ID NO: 14 - DNA, 20 bp, Artificial Sequence
  Oligonucleotide
ggtatccagggactgaagca

SEQ ID NO: 15 - DNA, 20 bp, Artificial Sequence
  Oligonucleotide
cgattgtcagcagagacgaa

SEQ ID NO: 16 - DNA, 20 bp, Artificial Sequence
  Oligonucleotide
tccatctgtaaccgtcgtca

SEQ ID NO: 17 - DNA, 20 bp, Artificial Sequence
  Oligonucleotide
gacgagtttatccgcgagag

SEQ ID NO: 18 - DNA, 20 bp, Artificial Sequence
  Oligonucleotide
acacgccatagttcggtttc

SEQ ID NO: 19 - DNA, 20 bp, Artificial Sequence
  Oligonucleotide
catgcttgcttcgatgttgt

SEQ ID NO: 20 - DNA, 20 bp, Artificial Sequence
  Oligonucleotide
cttgatccccgtggtaaaaa

SEQ ID NO: 21 - DNA, 20 bp, Artificial Sequence
  Oligonucleotide
cggtcaagaaggagaagacg

SEQ ID NO: 22 - DNA, 20 bp, Artificial Sequence
  Oligonucleotide
cttcgccgggtctctaatct

SEQ ID NO: 23 - DNA, 21 bp, Artificial Sequence
  Oligonucleotide
ggtgctaagaagaggaagaat

SEQ ID NO: 24 - DNA, 21 bp, Artificial Sequence
  Oligonucleotide
ctccttctttctggtaaacgt

SEQ ID NO: 25 - DNA, 20 bp, Artificial Sequence
  Oligonucleotide
ccatgaccgatcaactaacg

SEQ ID NO: 26 - DNA, 20 bp, Artificial Sequence
  Oligonucleotide
gaaccctggctctttttcaa

SEQ ID NO: 27 - DNA, 22 bp, Artificial Sequence
  Oligonucleotide
tcatacgtgtcagaacgagaca

SEQ ID NO: 28 - DNA, 20 bp, Artificial Sequence
  Oligonucleotide
cagaccttgtggccttgact

SEQ ID NO: 29 - DNA, 20 bp, Artificial Sequence
  Oligonucleotide
atgaaaagaattccggtcca

SEQ ID NO: 30 - DNA, 23 bp, Artificial Sequence
  Oligonucleotide
ttggaaaagaaatgagacatcaa

SEQ ID NO: 31 - DNA, 20 bp, Artificial Sequence
  Oligonucleotide
aaattcccttcccaaatggt

SEQ ID NO: 32 - DNA, 20 bp, Artificial Sequence
  Oligonucleotide
acaagcgaaccaaggaaaaa

SEQ ID NO: 33 - DNA, 20 bp, Artificial Sequence
  Oligonucleotide
ccaatcacaacattgcttcg

SEQ ID NO: 34 - DNA, 20 bp, Artificial Sequence
  Oligonucleotide
aaacacgtgcgttttatcca

SEQ ID NO: 35 - DNA, 25 bp, Artificial Sequence
  Oligonucleotide
cgtttcgctttccttagtgttagct

SEQ ID NO: 36 - DNA, 27 bp, Artificial Sequence
  Oligonucleotide
agcgaacggatctagagactcaccttg

SEQ ID NO: 37 - DNA, 21 bp, Artificial Sequence
  Oligonucleotide
cgtcgagaaatgttgtgtgtg

SEQ ID NO: 38 - DNA, 23 bp, Artificial Sequence
  Oligonucleotide
tgggtcctataaccctgtaacat

SEQ ID NO: 39 - DNA, 27 bp, Artificial Sequence
  Oligonucleotide
tttataaaataataggtcaacctcgaa

SEQ ID NO: 40 - DNA, 25 bp, Artificial Sequence
  Oligonucleotide
catgtattatccactgttttcctct

SEQ ID NO: 41 - DNA, 20 bp, Artificial Sequence
  Oligonucleotide
tgcatcggtgttagaaggaa

SEQ ID NO: 42 - DNA, 21 bp, Artificial Sequence
  Oligonucleotide
ttgttgagtgggaaaaggttg

SEQ ID NO: 43 - DNA, 27 bp, Artificial Sequence
  Oligonucleotide
tctacaatactaccaaacagaaccaaa

SEQ ID NO: 44 - DNA, 20 bp, Artificial Sequence
  Oligonucleotide
gagaggaggatttggggaat

SEQ ID NO: 45 - DNA, 22 bp, Artificial Sequence
  Oligonucleotide
gattgagctcatagtcccgttt

SEQ ID NO: 46 - DNA, 24 bp, Artificial Sequence
  Oligonucleotide
ttttcttatttcgtgtcactttgg

SEQ ID NO: 47 - DNA, 23 bp, Artificial Sequence
  Oligonucleotide
tttgtttgatcagtctttgtcca

SEQ ID NO: 48 - DNA, 21 bp, Artificial Sequence
  Oligonucleotide
ttgcttgggttttaagtttgg

SEQ ID NO: 49 - DNA, 22 bp, Artificial Sequence
  Oligonucleotide
cactctggccctatacttcctt

SEQ ID NO: 50 - DNA, 20 bp, Artificial Sequence
  Oligonucleotide
ttgaccaaatgaccaaacca

SEQ ID NO: 51 - DNA, 20 bp, Artificial Sequence
  Oligonucleotide
attcagccgccttacacaaa

SEQ ID NO: 52 - DNA, 20 bp, Artificial Sequence
  Oligonucleotide
tccaaggaaaagcaagcaat

SEQ ID NO: 53 - DNA, 20 bp, Artificial Sequence
  Oligonucleotide
acaaaccgaaccgcttttta

SEQ ID NO: 54 - DNA, 20 bp, Artificial Sequence
  Oligonucleotide
agaacaaaccgctgcaaact

SEQ ID NO: 55 - DNA, 20 bp, Artificial Sequence
  Oligonucleotide
gccggctatgctataacgaa

SEQ ID NO: 56 - DNA, 20 bp, Artificial Sequence
  Oligonucleotide
tatgacgctctccacgtttg